(19) United States
(12) Patent Application Publication
(10) Pub. No.: US 2025/0380961 A1
(43) Pub. Date: Dec. 18, 2025

(54) TISSUE ABLATION DEVICE WITH EXPANDABLE ELEMENT FOR DEVICE ARTICULATION

(71) Applicant: Gynesonics, Inc., Redwood City, CA (US)

(72) Inventors: Harry KWAN, San Francisco, CA (US); Katherine GEARST, Lincoln Park, NJ (US); Jiayu CHEN, Palo Alto, CA (US)

(21) Appl. No.: 18/879,143

(22) PCT Filed: Jun. 26, 2023
(86) PCT No.: PCT/US2023/069094

§ 371 Date: Dec. 26, 2024

Related U.S. Application Data
(60) Provisional application No. 63/355,876, filed on Jun. 27, 2022.

(51) Int. Cl. A61B 17/32; A61B 8/00; A61B 8/12; A61B 10/02; A61B 10/04; A61B 17/00; A61B 17/42

(52) U.S. Cl. CPC A61B 17/320016; A61B 8/12; A61B 8/445; A61B 8/4466; A61B 10/0275; A61B 10/0291; A61B 2010/045; A61B 2017/00557; A61B 2017/320069; A61B 2017/320071; A61B 2017/4216

(57) ABSTRACT

Aspects of the present disclosure include an inflatable balloon or other expandable element to articulate a working end of a tissue ablation probe in tissue ablation systems, devices, and methods.

### CROSS-REFERENCE TO RELATED APPLICATION AND INCORPORATION BY REFERENCE

This application claims the benefit of U.S. Provisional Application No. 63/355,876, filed Jun. 27, 2022, the entirety of which is hereby incorporated by reference.

All publications, patents, and patent applications mentioned in this specification are herein incorporated by reference to the same extent as if each individual publication, patent, or patent application was specifically and individually indicated to be incorporated by reference.

### BACKGROUND OF THE INVENTION

### Technical Field

The present disclosure relates to medical systems, devices, and methods, particularly for uterine fibroid ablation. More particularly, the present disclosure relates to imaging components in use with therapeutic and diagnostic instruments.

### Description of the Related Art

Current systems, devices, and methods for imaging and/or for therapeutic procedures may be less than ideal in at least some respects. For example, many current devices may have limited flexibility for use in a variety of diagnostic and therapeutic procedures. For example, many current devices may risk injuring a patient during insertion and/or removal, or may be difficult to navigate to a target location.

Additionally or alternatively, current systems, devices, and methods for diagnosing and providing therapy may be less than ideal in at least some other respects. For example, in procedures where more than one instrument may be required, multiple instruments may need to be inserted or removed from a patient lumen, and these additional steps of insertion and removal may increase injury risk for the patient. Additionally or alternatively, many current methods may require removal of an imaging component many times during a single procedure, and the removal of the imaging component may limit the ability to continually and steadily view the surgical field during the procedure. Moreover, many current devices may be limited in their ability to articulate within a cavity or lumen in the body of a patient, which can limit the ability of instruments (e.g., imaging and/or procedural instruments) to reach target tissue.

In light of the above, improved systems, devices, and methods for imaging a surgical field are desired. Such systems, devices, and methods would address at least some of the drawbacks above and would, for example, be easier to be used for a greater variety of therapeutic and diagnostic procedures.

### SUMMARY

The embodiments disclosed herein each have several aspects no single one of which is solely responsible for the disclosure's desirable attributes. Without limiting the scope of this disclosure, its more prominent features will now be briefly discussed. After considering this discussion, and particularly after reading the section entitled “Detailed Description,” one will understand how the features of the embodiments described herein provide advantages over existing systems, devices and methods.

The present disclosure relates to medical systems, devices, and methods, particularly for but not limited to uterine fibroid ablation. Embodiments of the present disclosure provide an expandable element for device articulation. Such an expandable element can increase ease of use and device deployment and positioning. Such expandable element(s) can also maintain image quality by keeping the imaging transducer of the tissue ablation device consistently in contact with tissue.

In some embodiments, disclosed herein is a tissue ablation device comprising: a device shaft; an imaging transducer provided at a distal end of the device shaft providing an ultrasound tip; and an expandable element configured to articulate the ultrasound tip.

In the above tissue ablation device or in other embodiments as described herein, one or more of the following features may also be provided. In some embodiments, the imaging transducer is pivotable between a straight position in which the imaging transducer forms a straight angle with the device shaft, and a pivoted position in which the imaging transducer is angled relative to the device shaft. In some embodiments, the ultrasound tip of the tissue ablation device comprises a spring-loaded tip, the spring-loaded tip configured to apply a force to restore the imaging transducer from a pivoted position toward the straight position. In some embodiments, the expandable element is directly coupled to the device shaft. In some embodiments, the imaging transducer is configured to image tissue in a forward direction, and wherein the expandable element is configured to expand in a backwards direction, the backwards direction being substantially opposite from the forward direction. In some embodiments, the expandable element is a balloon. In some embodiments, the balloon comprises a plurality of chambers, wherein each chamber can be controlled individually. In some embodiments, the balloon is configured to be expanded by being filled with a filler fluid. In some embodiments, the filler fluid is gaseous. In some embodiments, the filler fluid is a high temperature filler fluid. In some embodiments, the balloon is configured to expand between the imaging transducer and tissue being imaged by the imaging transducer, such that the balloon acts as an acoustic standoff to couple the imaging transducer with the tissue. In some embodiments, the expandable element is coupled to the device shaft proximal to the imaging transducer. In some embodiments, the expandable element is an unwinding spring. In some embodiments, the device shaft comprises a straight shaft. In some embodiments, the tissue ablation device is configured for uterine fibroid ablation.

### BRIEF DESCRIPTION OF THE DRAWINGS

Certain features of this disclosure are described below with reference to the drawings. The illustrated implementations are intended to illustrate, but not to limit, the implementations. Various features of the different disclosed implementations can be combined to form further implementations, which are part of this disclosure.

FIG. 1A shows a perspective view of an imaging component, in accordance with some embodiments.

FIG. 1B shows a side, cross-sectional view of the imaging component of FIG. 1A, in accordance with some embodiments.

FIG. 1C shows a side, cross-section view of an imaging component having a shaft with a circular cross-section, in accordance with some embodiments.

FIG. 1D shows a side, cross-sectional view of an imaging component with edges bent inward towards the interior of the cavity, in accordance with some embodiments.

FIG. 1E shows a magnified, perspective view of a distal end of the imaging component of FIG. 1A comprising a cavity, in accordance with some embodiments.

FIG. 2A shows a magnified, perspective view of a distal end of the imaging component of FIG. 1A with a tissue collector instrument disposed within the shaft of the imaging component, in accordance with some embodiments.

FIG. 2B shows a side, cross-sectional view of the imaging component of FIG. 1A with a biopsy instrument disposed within the shaft of the imaging component, in accordance with some embodiments.

FIG. 2C shows a magnified, perspective view of a distal end of the imaging component of FIG. 1A with a radiofrequency ablation instrument disposed within the shaft of the imaging component, in accordance with some embodiments.

FIG. 2D shows a top view of the imaging component of FIG. 1A with a drug delivery instrument disposed within the shaft of the imaging component, in accordance with some embodiments.

FIG. 2E shows a side, cross-sectional view of the imaging component of FIG. 1A with a needle disposed within the shaft of the imaging component, in accordance with some embodiments.

FIG. 3A shows an assembly view of an imaging system comprising the imaging component of FIG. 1A and an optical scope instrument, in accordance with some embodiments.

FIG. 3B shows an assembly view of the imaging system of FIG. 3A illustrating an attachment mechanism of the system, in accordance with some embodiments.

FIG. 4 shows a magnified, perspective view of a shaft of the imaging component of FIG. 1A wherein the shaft of the imaging component is flexible, in accordance with some embodiments.

FIG. 5A illustrates a perspective view of a system for diagnosing and/or providing therapy, including an imaging component configured to be removably coupled to multiple therapeutic and/or diagnostic instruments, in accordance with some embodiments. FIG. 5A shows the imaging component and the therapeutic and/or diagnostic instrument being separated.

FIG. 5B illustrates a perspective view of the system of FIG. 5A, with the therapeutic and/or diagnostic instrument being in a ready position to be removably coupled to the imaging component, in accordance with some embodiments.

FIG. 5C illustrates a perspective view of the system of FIG. 5A, with the therapeutic and/or diagnostic instrument being removably coupled to the imaging component, in accordance with some embodiments.

FIG. 6 shows a schematic of an imaging system comprising a digital processing device and a display visible to a user, in accordance with some embodiments.

FIG. 7A shows a schematic of the imaging component of FIG. 1A positioned within a uterus to image tissue thereof, in accordance with some embodiments.

FIG. 7B shows a surgical field image captured as in FIG. 7A that would be visible on a display, showing safety and treatment boundaries, in accordance with some embodiments.

FIG. 7C shows a surgical field image combining both a virtual image showing safety and treatment boundaries and the physical presence of an introducer, in accordance with some embodiments.

FIG. 7D shows a surgical field image combining both a virtual image showing safety and treatment boundaries as well as the physical presence of an introducer and needle electrodes, in accordance with some embodiments.

FIGS. 8A-8C show a series of schematics of a tissue ablation device that depict a method by which an expandable element can be expanded within a uterus, the device articulated to access a sidewall of the uterus, in accordance with some embodiments.

FIG. 9 shows a schematic of a tissue ablation device positioned within a uterus with an expandable element in an expanded configuration, articulated to access the fundus of the uterus, in accordance with some embodiments.

### DETAILED DESCRIPTION OF PREFERRED EMBODIMENTS

Various features and advantages of this disclosure will now be described with reference to the accompanying figures. The following description is merely illustrative in nature and is in no way intended to limit the disclosure, its application, or uses. This disclosure extends beyond the specifically disclosed implementations and/or uses and obvious modifications and equivalents thereof. Thus, it is intended that the scope of this disclosure should not be limited by any particular implementations described below. The features of the illustrated implementations can be modified, combined, removed, and/or substituted as will be apparent to those of ordinary skill in the art upon consideration of the principles disclosed herein. Furthermore, implementations disclosed herein can include several novel features, no single one of which is solely responsible for its desirable attributes or which is essential to practicing the systems, devices, and/or methods disclosed herein.

Certain embodiments of the present disclosure are directed to imaging and/or therapeutic devices, and associated methods and systems, that incorporate an expandable element for improved device articulation. Examples of these devices, methods and systems are described in the examples below, followed by examples of how these devices, methods and systems may be applied to uterine fibroid ablation. However, the improvements described herein are not limited to uterine fibroid ablation, and may be incorporated into any of the imaging and/or therapeutic devices described herein.

### Examples of Imaging and/or Therapeutic Devices

Embodiments of the present disclosure provide an imaging component comprising a cavity extending across (e.g., along) the length of a shaft, wherein the cavity may be configured to removably receive at least one of a plurality of different instruments. In some embodiments, the cavity of the imaging component may be partially open to an exterior of the shaft. The imaging component may comprise an imaging transducer at the distal end of the shaft. Additionally, the shaft of the imaging component may be configured such that additional therapeutic and/or diagnostic instruments/attachments may be removed and/or received and/or inserted during a medical procedure without disturbing the imaging component. Additionally or alternatively, the imaging component may remain in situ while the therapeutic and/or diagnostic instrument is received and/or removed. In some embodiments, the imaging component may be used without an additional therapeutic and/or diagnostic instrument coupled thereto. In some embodiments, the imaging component may be inserted and/or removed from a patient lumen without the presence of a therapeutic and/or diagnostic instrument. Such an imaging component may be used during a medical procedure such as, for example, non-invasive, minimally invasive, and/or laparoscopic surgery.

Embodiments of the present disclosure may improve upon existing methods for imaging and treating a lesion in a tissue tract for procedures where multiple instruments may be required to diagnose and/or provide therapy during a single procedure. For example, an imaging component may be used for diagnosis; then a biopsy attachment may be inserted for a pathology sample; then an ablation attachment may be inserted for ablating any lesions; and then a further attachment or instrument may be inserted to perform additional procedures such a deliver drugs, implants, and/or therapeutic and/or diagnostic agents. The imaging component of the present disclosure may facilitate the insertion and removal of medical instruments by providing a shaft with atraumatic edges and a cavity configured to receive a plurality of different instruments. Additionally or alternatively, the imaging component may be used independently of an additional instrument or attachment. In such embodiments, the edges of the cavity may be smooth or rounded such that the edges may not catch on the patient tissue when used alone.

The cavity of an imaging component may improve upon existing methods for imaging and treatment by providing a cavity of an imaging component which may be easier to clean than a component with a closed cavity or lumen. The cavity of an imaging component may improve on existing methods for imaging and treatment by facilitating manufacture of the imaging component. Embodiments of the present disclosure may lower treatment cost by providing an imaging component with a disposable tube. Embodiments of the present disclosure may lower treatment costs by providing a reusable imaging component with a cavity into which disposable instruments may be inserted. Embodiments of the imaging component may provide a shaft which aligns the instrument with the ultrasound image at all times. Embodiments of the present disclosure may accommodate various instruments with different sizes and shapes. Embodiments of the present disclosure may provide a scale or position information to assist insertion of an instrument.

The systems and methods of the present disclosure may be particularly useful in the treatment of fibroids in a patient uterus. The imaging component may be deployed transvaginally and transcervically into the uterus, or in other cases, laparoscopically into and through an exterior of the uterus or other organ or tissue tract. The imaging component may be used in conjunction with an additional instrument such as a biopsy needle; a tissue ablation element, such as for example a radiofrequency ablation element, an ultrasonic ablation element, a heat-based ablation element, a cryoablation element, etc.; and/or other instrument suitable to be disposed within the cavity of the imaging component. Additionally or alternatively, the additional instrument may be used to deliver drugs, implants, or other therapeutic agents to the tissue to be treated. Additionally or alternatively, the tissue ablation element may comprise embodiments or variations of the needle/tine assemblies of commonly assigned U.S. Pat. Nos. 8,206,300, 8,262,574, and 8,992,427, the contents of which are incorporated herein by references.

Embodiments of the present disclosure may improve upon at least some of the systems and methods of the commonly assigned references by providing a shaft of an imaging component with atraumatic edges to enable use of the imaging component alone. In some embodiments, embodiments of the present disclosure may improve upon the ability to remove and/or receive an additional instrument by providing an imaging system without an attachment mechanism located in at least the portion of the system to be positioned in situ. In such an embodiment, the imaging component shaft may be non-cylindrically symmetric (e.g., oval or rectangular in cross-section) in order to reference the rotation of the additional instrument relative to the imaging component shaft. In some embodiments, the present disclosure may additionally or alternatively provide a shaft of an imaging component with a small angled portion to minimize damage risk to a surface of an imaging transducer surface by an instrument. Additionally or alternatively, the imaging component may comprise a disposable tube inserted within the cavity to provide, among many possible purposes, a working channel for inserting additional instruments with different diameters and making the system easier to clean.

The imaging components described herein may be used in a surgical procedure to provide a real time image of a target structure to be treated, including projecting safety and treatment boundaries as described in commonly assigned U.S. Pat. Nos. 8,088,072 and 8,262,577, the contents of which are incorporated by reference. The imaging components described herein may be useful for both imaging and treating uterine fibroids as described in commonly assigned U.S. Pat. No. 7,918,795, which is incorporated herein by reference. Other commonly assigned patents and published applications describing probes useful for treating uterine fibroids which may be used with the imaging components described herein include U.S. Pat. Nos. 7,815,571, 7,874,986, 8,506,485, 9,357,977, and 9,517,047, which are incorporated herein by references. Additional, commonly assigned patent applications describing systems for establishing and adjusting displayed safety and treatment zone boundaries which may be used in conjunction with the imaging components described herein include: U.S. Pat. Pub. No. 2014/0073910 (now U.S. Pat. No. 9,861,336); U.S. Pat. Pub. No. 2019/0350648; U.S. Pat. No. 8,992,427; U.S. Pat Pub. No. 2018/0132927 (now U.S. Pat. No. 11,219,483); and P.C.T. Pub. No. WO2018/089523, which are each incorporated herein by reference. Commonly assigned P.C.T. Pub. No. WO2018/089523, further describes mapping and planning system which may be used in conjunction with the imaging components described herein, is also incorporated herein by reference.

In some embodiments, the systems and methods of the present disclosure may provide an imaging component to be used in a variety of diagnostic and therapeutic procedures. Some embodiments may provide methods and systems to perform therapy or diagnosis on a volume of tissue. A volume of tissue may comprise a patient organ. A patient organ or bodily cavity may comprise for example: muscles, tendons, a mouth, a tongue, a pharynx, an esophagus, a stomach, an intestine, an anus, a liver, a gallbladder, a pancreas, a nose, a larynx, a trachea, lungs, a kidneys, a bladder, a urethra, a uterus, a vagina, an ovary, testes, a prostate, a heart, an artery, a vein, a spleen, a gland, a brain, a spinal cord, a nerve, etc. Some embodiments provide systems and methods suitable for laparoscopic surgery. Some embodiments provide systems and methods suitable for non-invasive surgery. Some embodiments provide systems and methods suitable for minimally invasive surgery. Some embodiments provide systems and methods suitable for robotic or robot assisted surgery.

Certain embodiments of this present disclosure are configured to create a mechanism for directing the straight portion of an imaging and/or treatment device as described herein (such as the treatment device of the Sonata System available from Gynesonics, Inc. of Redwood City, CA) at a target fibroid while serving as the articulation mechanism for the ultrasound tip, in replacement of or alternative to the lever and pushrod elements of the device. An articulating mechanism controlling the pitch angle of the ultrasound transducer tip may be manually actuated to one of fixed positions and locked in place. Once this step is complete, the user may orient the device to direct the straight shaft directly through the widest portion of the target fibroid in order to place the introducer into the mass.

Reference will now be made in detail to various embodiments, examples of which are illustrated in the accompanying drawings. In the following detailed description, numerous specific details are set forth in order to provide a thorough understanding of the invention and the described embodiments. However, the invention is optionally practiced without these specific details. In other instances, well-known methods, procedures, components, and circuits have not been described in detail so as not to unnecessarily obscure aspects of the embodiments.

It will be understood that, although the terms “first,” “second,” etc. are optionally used herein to describe various elements, these elements should not be limited by these terms. These terms are only used to distinguish one element from another. For example, a first instrument could be termed an instrument sensor, and, similarly, a second instrument could be termed a first instrument, without changing the meaning of the description, so long as all occurrences of the “first instrument” are renamed consistently and all occurrences of the second instrument are renamed consistently. The first instrument and the second instrument are both instruments, but they are not the same instrument.

The terminology used herein is for the purpose of describing particular embodiments only and is not intended to be limiting of the claims. As used in the description of the embodiments and the appended claims, the singular forms “a”, “an” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, the term “if” is optionally construed to mean “when” or “upon” or “in response to determining” or “in accordance with a determination” or “in response to detecting,” that a stated condition precedent is true, depending on the context. Similarly, the phrase “if it is determined [that a stated condition precedent is true]” or “if [a stated condition precedent is true]” or “when [a stated condition precedent is true]” is optionally construed to mean “upon determining” or “in response to determining” or “in accordance with a determination” or “upon detecting” or “in response to detecting” that the stated condition precedent is true, depending on the context.

For ease of explanation, the figures and corresponding description below may be described below with reference to uterine imaging, specifically, in conjunction with the diagnosis and ablation and/or treatment of uterine fibroids. However, one of skill in the art will recognize that a similar imaging component may be used with similar instruments in other therapeutic applications for example: instruments for tissue biopsy, for drug delivery, for fluid infusion and/or aspiration, and for the treatment of cancers, tumors, fibroids, and other masses, malignant or benign, in any suitable bodily lumen.

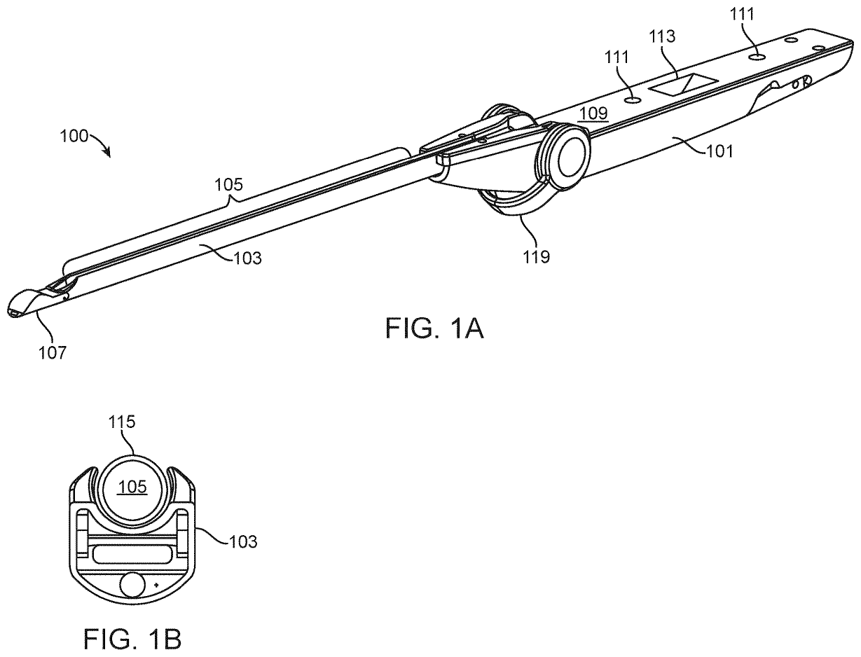

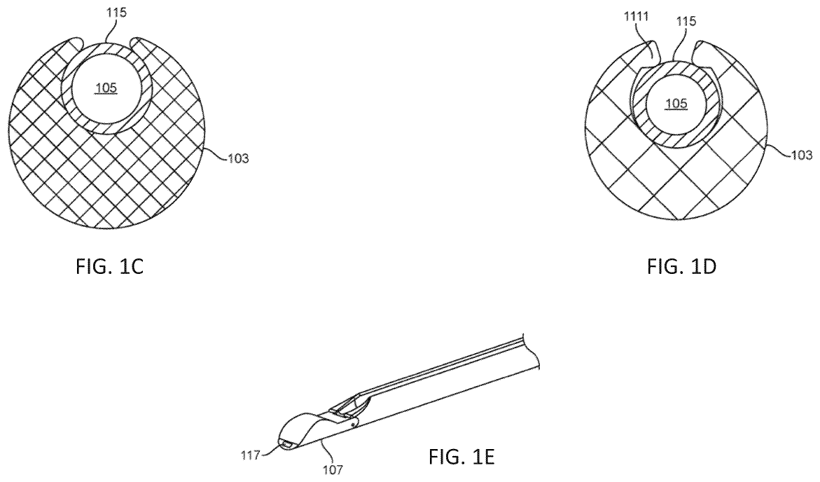

FIG. 1A shows an illustration of an imaging component 100, in accordance with some embodiments. Imaging component 100 may comprise a handle portion 101 connected to an imaging shaft 103. At the distal end of imaging shaft 103 may be coupled an imaging transducer 107. The imaging shaft 103 may comprise a proximal end and a distal end with a cavity 105 extending across the length of the shaft 103 from the proximal end towards the distal end. The cavity 105 may be at least partially open to the exterior of the shaft 103. For example, a side, or wall of the cavity 105 may comprise an elongated opening in communication with the exterior of the shaft 103. The elongated opening may be in communication with the exterior of the shaft 103 at least partially along the length of the shaft 103. In some embodiments, an edge of the elongated opening may be bent towards an interior of the cavity 105 of the shaft 103 (for example, see FIG. 1D further described below). The length of the shaft 103 may be sufficiently long to fully access the uterus of a patient while the handle portion 101 remains exterior to the patient. Additionally or alternatively, the shaft 103 may comprise a length significantly longer than the distance sufficient to fully access a patient uterus. The side opening may be open along the full length of the shaft 103 or it may be open only partially along the length of the shaft 103. The side opening may be open, for example, for greater than three-fourths the length of the shaft 103, for greater than half the length of the shaft 103, or for greater than one quarter the length of the shaft 103. The cavity 105 may be configured to receive at least one of a plurality of different additional instruments or attachments, such that a first instrument may be received by the cavity 105, the first instrument may be removed from the cavity 105, and a second instrument may be received by the cavity 105.

The handle portion 101 may be one part of a two-part handle such that when a first instrument or a second instrument is received the two handle portions may combine to form a single handle. The inside face of the handle portion 109 may comprise alignment elements 111 such that a first part and a second part of the handle may be reproducibly aligned with respect to one another after changing instruments. The alignment elements 111 may be configured such that a first part and a second part may be sufficiently secured with respect to one another to use the two handle portions as a single handle. In some embodiments, the alignment elements 111 may comprise magnets. In other embodiments, alignment elements 111 may comprise for example: latches, hooks, or any other mechanism suitable to removably combine a two-part handle. The handle portion may additionally comprise a positioning element 113, such as a slot to accommodate a complementary protrusion or other element on the opposite handle portion, in order to provide a more secure reference between parts of the two-part handle. The positioning element 113 may comprise a mechanical feature to secure the instrument relative to the imaging component 100 by limiting translation of the instrument on the axis of the shaft 103 of the imaging component.

In other embodiments, imaging component 100 may be configured to be used with an instrument which does not have a handle portion. In such embodiments, the handle portion 101 of the imaging component 100 is sufficient to be used alone to guide the imaging component during a procedure. In some embodiments, imaging component 100 may have a scale or a guide on the inside face of the handle portion 109 in order to gauge the insertion depth of an instrument. In other embodiments, the imaging component 100 may be used without an instrument. In some embodiments, a scale may facilitate embodiments where the instrument does not have a handle. In other embodiments, a scale may facilitate the insertion of a component of the instrument in embodiments where the instrument has a handle.

FIG. 1B shows a cross-sectional view of an imaging component 100, in accordance with some embodiments. The body of the shaft 103 may comprise internal structure in order to carry electronics or other associated components to control the imaging transducer 107. The shaft 103 may also comprise a wire system or other flex mechanism in order to allow the shaft 103 to controllably bend, flex, or deflect the distal end of the shaft 103. The shaft 103 may comprise a channel or duct to direct fluid (e.g., water, saline, etc.) to a distal end of the shaft 103 and onto a tissue surface. Imaging shaft 103 may be round in cross-section or take a shape with sufficiently softened, chamfered, rounded, or beveled edges such that the edges may be atraumatic to a patient opening during insertion or removal of an imaging component 100 with or without an instrument. Shaft 103 may additionally comprise a smooth exterior surface. Shaft 103 may be made of a material such that the surface may be deformable to allow the shaft 103 to bend or adapt to the shape of a bodily lumen.

The cavity 105 of imaging shaft 103 may be configured to slidably receive one or more of a plurality of instruments. In some embodiments, the cavity 105 may be defined by an exterior surface of the shaft 103. In some embodiments, the cavity 105 may be partially open along a wall, such that the cavity 105 may be in communication with the exterior of the shaft 103. The opening may be sufficiently closed to provide structural support such that when the imaging component 100 may be inserted into a patient bodily lumen, the opening of the lumen may not be significantly disturbed by the insertion or removal of an instrument. Optionally, the exterior surface of the shaft 103 may comprise only atraumatic edges. The cavity 105 of imaging shaft 103 may be sufficiently open such that when instruments of different sizes may be received or inserted into the cavity, the cavity may allow some distortion of the cavity opening. The cavity 105 may facilitate cleaning of the imaging component.

FIG. 1C shows a cross-section view of an imaging component having a shaft 103 with a circular cross-section, in accordance with some embodiments. The imaging component of FIG. 1C may be sufficiently circular in cross-section such that the imaging component may be rotated without disturbing a patient lumen. FIG. 1D shows a cross-sectional view of an imaging component with edges bent inward towards the interior of the cavity 105, in accordance with some embodiments. The inward bent edges 1111 of a cavity may serve to support the opening of a bodily lumen such that the shaft 103 may be inserted or removed atraumatically from a bodily lumen with or without an instrument.

While the cavity 105 of the shaft 103 in the illustrated example may define a circular cross sectional geometry, in other embodiments the cavity may be elliptical or any other geometric shape with sufficiently softened, rounded, or beveled edges and corners such that insertion or removal of the shaft may not damage the patient bodily lumen. In some embodiments, the cavity 105 may be non-cylindrically symmetric. In some embodiments, the cavity 105 may be asymmetrical to provide an axis for alignment of the instrument within. The cavity 105 may be open for less than three-quarters its perimeter in cross-section, additionally or alternatively, the cavity may be open for less than half its perimeter, less than a quarter its perimeter, and less than one eighth its perimeter. In other embodiments, the cavity 105 of the shaft 103 of the imaging component may be closed to the exterior of the shaft, and an instrument may be slidably inserted fully interior to the shaft of the imaging component.

In some embodiments, the cavity 105 may comprise a substantially uniform cross sectional area along the shaft 103. In other embodiments, a portion of the length of the shaft 103 may have a different cross section than another portion of the length of the shaft. In an example, the proximal portion of the shaft 103 may be asymmetric to provide an axis for alignment of an instrument and the distal portion of the shaft may have a circular cross sectional area. In another embodiment, the cavity 105 tapers toward the end of the shaft 103. In such an example, the taper may facilitate feeding an instrument into the cavity 105. In some embodiments, the cross sectional area of the cavity 105 may narrow in diameter to allow greater flexibility of the distal end of the shaft 103.

In some embodiments, imaging shaft 103 may additionally comprise a tube 115 to be positioned at the cavity 105 of imaging shaft 103. Tube 115 may comprise a lumen. The lumen of tube 115 may be configured to slidably receive one or more of a plurality of instruments. Tube 115 may be aligned in parallel with the shaft 103 of the imaging component, such that an additional instrument/attachment may be slidably received by the tube. Subsequently, the tube 115 may slidably receive the additional instrument/attachment after it has been aligned to be in parallel with the shaft 103 of the imaging component. In some embodiments, the tube 115 may be disposable. In some embodiments, the tube 115 may be reusable such as by being un-coupled from the imaging shaft 103, washed, and autoclaved. Tube 115 may have an exterior surface wherein the surface is substantially in contact with the inner wall of cavity 105. Tube 115 may have an interior surface of a different geometry to the outer surface configured to receive one or more of a plurality of instruments. In some embodiments, a second tube (not shown) may be removably inserted into the first tube 115 and the second tube may have a different inner lumen geometry than the first, thereby aiding in the insertion of one or more of a plurality of instruments. In some embodiments, the tube 115 may be rotated relative to the imaging component. In some embodiments, the tube 115 may fully rotate relative to the imaging component in either direction under the control of a user within the shaft 103 of the imaging component. In some embodiments, the tube 115 may be internally or externally lubricated to facilitate insertion or removal of an instrument.

The tube 115 may be inserted into the bodily lumen in situ with the imaging component yet advanced therein. Additionally or alternatively, the tube 115 may be inserted into the shaft 103 of the imaging component prior to insertion of the imaging component into the bodily lumen. The tube 115 may have sufficient structural integrity to support a bodily lumen during insertion of the imaging component without an instrument. When an additional instrument is inserted into the tube 115 or the tube 115 is inserted into the imaging component in situ, disruption to the bodily lumen may be minimized. The tube 115 may be made of a material that can be sterilized. The tube 115 may be made of a material that may be of low enough cost that it may be disposed of after a single use. Exemplary materials for a disposable tube may comprise polyimide, PTFE, Urethanes and thermoplastics like Pebax or Nylon, etc. Tube 115 may be made of a material comprising sufficient elasticity in order to adapt to an instrument of a size somewhat larger or smaller than the perimeter of the tube. In embodiments where the cavity 105 is not circular, the tube 115 may take the shape of the cavity or it may take another shape.

The tube 115 may lower treatment costs by facilitating insertion and/or removal of an additional instrument into the cavity 105 of the imaging component 100 and thereby preventing damage to the surface of the cavity 105 of the imaging component 100. The tube 115 may lower cost by facilitating cleaning of the cavity 105 of the imaging component 100. The tube 115 may lower cost of treatment by providing an inexpensive component which may act as an adapter for a variety of different therapeutic and/or diagnostic instruments/attachments, such as being provided in a variety of different inner geometries suitable for the different instruments/attachments but having a uniform outer geometry to be removably coupled to the same single imaging component 100. For example, a disposable tube with a smaller inner diameter may facilitate the insertion and control of a needle with a smaller outer diameter than the inner diameter of the shaft 103 of the imaging component.

FIG. 1E shows a magnified view of a distal end of the imaging component 100 comprising a cavity 105, in accordance with some embodiments. The distal end of the imaging component 100 may comprise an imaging transducer 107. The imaging transducer 107 may comprise an ultrasound transducer and/or a plurality of ultrasound transducers. The ultrasound transducer may operate at a frequency of 500 kHz, 1 MHz, 5 MHz, 10 MHz, 20 MHz, 100 MHz, or a range defined by any two of the preceding values. Some embodiments of the ultrasound transducer may comprise specifications of other transducers from the commonly assigned references incorporated herein.

In some embodiments, the distal end 117 of the imaging transducer 107 may additionally comprise a light emitting diode and/or a camera in order to provide images to a user. In such embodiments, the imaging component 100 may serve as an optical scope as well as an ultrasound imaging platform. The distal end 117 of the imaging transducer 107 may comprise optical components, such as an optic fiber, a relay lens, an objective lens, etc.

The imaging transducer 107 may be configured to be deflectable. The imaging transducer 107 may be configured to deflect relative to the longitudinal axis of the shaft 103 of the imaging component 100. In some embodiments, the distal end of an imaging component 100 comprises a hinge to facilitate deflection of an imaging transducer 107. The deflection of the imaging transducer 107 may be controlled by a deflection lever 119 on the handle portion 101 of the imaging component 100. The one or a plurality of imaging transducers 107 may be oriented by the deflection of the imaging transducer. The one or a plurality of imaging transducers 107 may be oriented by the deflection of the imaging transducer in order to facilitate maintaining the field of view of an image during a treatment. Additionally or alternatively, the imaging transducers 107 (e.g., ultrasound transducers) may be aligned radially and/or axially to image multiple views simultaneously. Deflection of the imaging transducer 107 may be induced in order to avoid obstruction of an instrument. Additionally or alternatively, deflection of the imaging transducer 107 may be used to deflect a flexible instrument within the cavity 105. The distal end of the shaft 103 may comprise an interlock system, similar to those in the incorporated references, in order to prevent the imaging transducer 107 from obstructing an instrument or being damaged by sharp edges of an instrument. Actuation of the deflection lever 119 may function in a manner similar to that described in U.S. Pat. No. 8,992,427, incorporated herein by reference. The deflection lever 119 may deflect the imaging transducer 107 by less than 45 degrees and additionally or alternatively, for example, less than 120 degrees, less than 90 degrees, less than 60 degrees, less than 30 degrees, less than 15 degrees, and less than 5 degrees.

The distal end of the imaging component 100 may comprise atraumatic edges in order to facilitate insertion of the imaging component with or without an instrument in the cavity 105. The distal end of the cavity 105 of the imaging component 100 may additionally or alternatively comprise a portion angled axially relative to the shaft 103, such that a distal end of an instrument may be deflected upward as it is pushed out the distal end of the cavity 105. The distal end of the cavity 105 of the imaging component 100 may comprise an angled portion with an angle of 3 to 45 degrees. The distal end of the cavity 105 of the imaging component 100 may comprise an angled portion with an angle at less than 45 degrees and additionally or alternatively, for example, less than 90 degrees, less than 60 degrees, less than 30 degrees, less than 15 degrees, and less than 5 degrees.

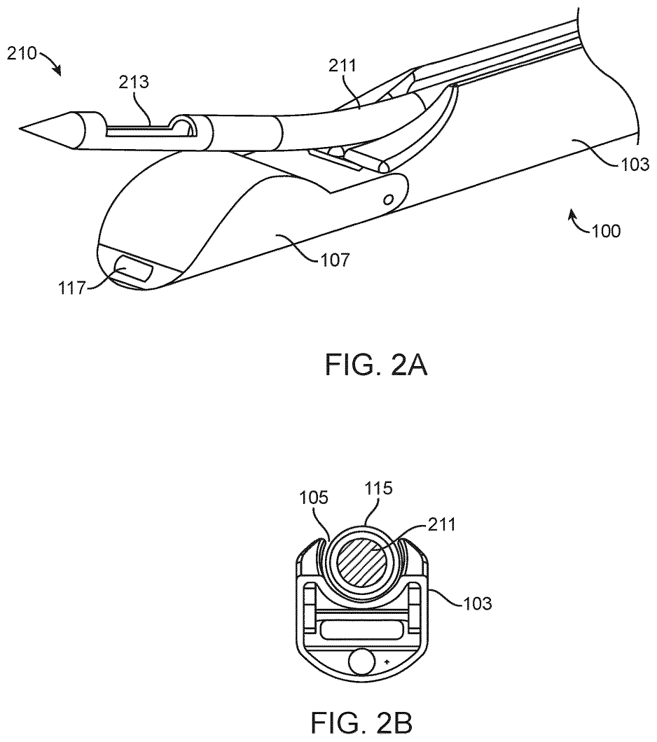

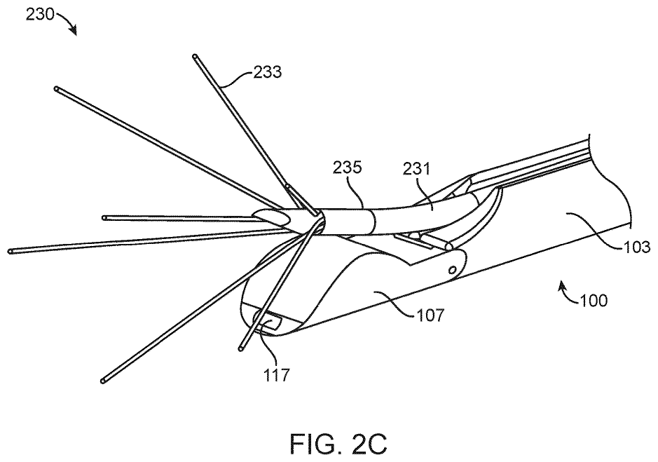

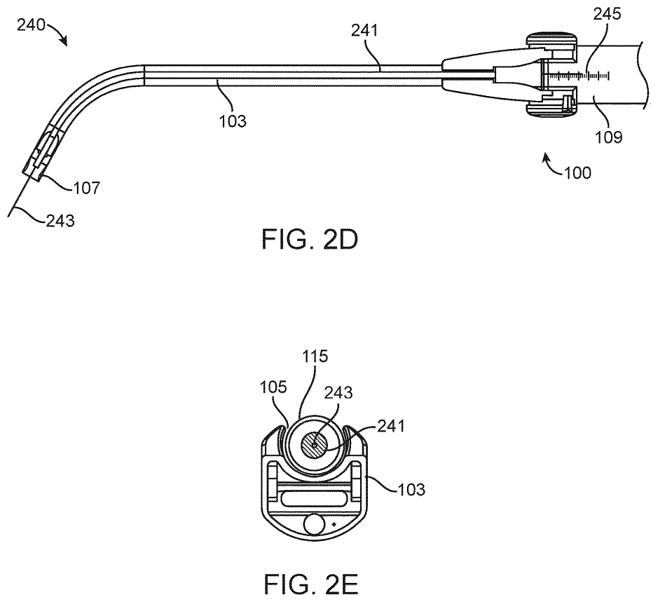

The cavity 105 of the imaging component 100 may be configured to slidably receive one or more of a plurality of instruments. In some embodiments, the imaging component 100 may be configured to receive one or a plurality of therapeutic or diagnostic instruments. In some embodiments, at least one of the plurality of different instrument may be a therapeutic or diagnostic instrument. In some embodiments, the instrument may comprise an instrument such as a biopsy needle; an optical scope; implantation device; therapy electrodes; a tissue ablation element, such as for example a radiofrequency ablation element, an ultrasonic ablation element, a heat-based ablation element, a cryoablation element, etc.; and/or other instrument suitable to be disposed within the cavity of the imaging component. Additionally or alternatively, the instrument may be used to deliver drugs or other therapeutic agents to the tissue to be treated. FIGS. 2A-2E show instruments which may be slidably received by the imaging component. One of ordinary skill in the art will recognize that many instruments, including those disclosed in the following figures, may be used with the imaging component disclosed herein.

FIG. 2A shows a magnified view of a distal end of the imaging component 100 with a tissue collector instrument 210 disposed within the shaft 103 of the imaging component 100, in accordance with some embodiments. The tissue collector instrument 210 may be used to extract tissue and/or cell pathology samples for examination by a medical professional to determine the extent of a disease. In some embodiments, the tissue collector instrument 210 may comprise a biopsy needle. The tissue collector instrument 210 may comprise a shaft 211 of a tissue collector instrument 210, which has a distal end and a proximal end. The shaft 211 of tissue collector instrument 210 may be configured to detach from a handle component of the instrument or may be configured to be used without a handle component such that the tissue collector instrument 210 may be disposable.

The shaft 211 of tissue collector instrument 210 may be made of a pliable and/or flexible material such that it may be deflected by the imaging transducer 107 and/or an angled portion within the cavity 105 of the shaft 103. In the illustrated example, a distal end of a shaft 211 of a tissue collector instrument 210 is deflected upward by an angled portion within the cavity 105 of the shaft 103. The distal end of a shaft 211 of a tissue collector instrument 210 may be deflected up in order to avoid damage of the imaging transducer 107, among other possible purposes. The distal end of the cavity 105 of the imaging component 100 may comprise a portion angled axially relative to the shaft 103, such that a distal end of an instrument (e.g., the tissue collector instrument 210) may be deflected upward as it is pushed out the distal end of the cavity 105. The distal end of the cavity 105 of the imaging component 100 may comprise an angled portion angled less than 45 degrees and additionally or alternatively, for example, less than 90 degrees, less than 60 degrees, less than 30 degrees, less than 15 degrees, and less than 5 degrees.

Additionally or alternatively, the shaft 103 of the imaging component 100 may comprise a wire system or other means to deflect the distal end of an instrument (e.g., the tissue collector instrument 210) such that a distal end of the instrument (e.g., the tissue collector instrument 210) does not damage the imaging transducer 107. The distal end of the tissue collector instrument 210 may comprise a slot or opening 213 into which tissue may be collected. In some embodiments, the tissue collector instrument 210 may rotate relative to the shaft 103. In some embodiments, the tissue collector instrument 210 may fully rotate relative to the shaft 103 in either direction under the control of a user within the shaft 103 of the imaging component 100 while the shaft 103 remains stationary, such that the slot or opening 213 may scrape, scoop, or otherwise collect tissue.

The shaft 211 of the tissue collector instrument 210 may be longer than the shaft 103 of the imaging transducer 107 such that the slot or opening 213 may collect tissue from deep inside the uterus or other body cavity. In some embodiments, the shaft 211 of the tissue collector instrument 210 may be two inches longer than the shaft of the imaging transducer 107. Additionally or alternatively for example, the shaft 211 of the tissue collector instrument 210 may be six inches longer, may be four inches longer, may be two inches longer, may be the same length, or may be within a range of any two of the preceding values.

FIG. 2B shows a cross-sectional view of an imaging component 100 with a tissue collector instrument 210 disposed within the shaft 103 of the imaging component 100, in accordance with some embodiments. The tissue collector instrument 210 may be disposed within a tube 115 disposed within the cavity 105 of the imaging component 100. Additionally or alternatively, tissue collector instrument 210 may be disposed within the cavity 105 of the imaging component 100 without the use of a tube. While the shaft 211 of the tissue collector instrument 210 in the illustrated example may define a circular geometry, in other embodiments, the shaft 211 of the tissue collector instrument 210 may be elliptical any other geometric shape such that the shaft 211 may be inserted or removed from the cavity 105 of the imaging component 100. In some embodiments, the shaft 211 of the tissue collector instrument 210 may be asymmetrical to provide an axis for alignment of the tissue collector instrument 210 within the cavity 105 of the imaging component 100. In some embodiments, the cavity 105 comprises a substantially uniform cross sectional area along the length of the shaft 103 of the imaging component 100. In other embodiments, the cross sectional area of the cavity 105 changes along the length of the shaft 103 of the imaging component 100 such as, for example, the proximal end of the shaft 103 may be asymmetric to provide an axis for alignment while the distal end of the shaft 103 may be circular.

FIG. 2C shows a magnified view of a distal end of the imaging component 100 with an ablation instrument 230 disposed within the shaft 103 of the imaging component 100, in accordance with some embodiments. The ablation instrument 230 may contain a needle assembly comprising an introducer 235 and, optionally, needle electrodes, or tines 233. The shaft 231 of the ablation instrument 230 may be deployed from the shaft 103 of an imaging component 100. Additionally or alternatively, the introducer 235 may be deployed from a lumen of a tube 115. The ablation instrument 230 may comprise one or more of, for example, a radiofrequency (RF) ablation element, an ultrasonic ablation element, a heat-based ablation element, a cryoablation element, and any other type of ablation elements known to one of ordinary skill in the art.

Ablation instrument 230 may be disposed within a tube 115 disposed within the cavity 105 of the imaging component 100. Additionally or alternatively, ablation instrument 230 may be disposed within the cavity 105 of the imaging component 100 without the use of a tube. While the shaft 231 of the ablation instrument 230 in the illustrated example may define a circular cross-sectional geometry, in other embodiments, the shaft 231 of the ablation instrument 230 may be elliptical or any other geometric shape such that the shaft 231 may be inserted or removed from the cavity 105 of the imaging component 100. In some embodiments, the shaft 231 of the ablation instrument 230 may be asymmetrical to provide an axis for alignment of the instrument within the cavity 105 of the imaging component 100.

The shaft 231 of the ablation instrument 230 may be made of a pliable and/or flexible material such that it may be deflected by the imaging transducer 107 and/or an angled portion within the cavity 105 of the shaft 103 of the imaging component 100. Additionally or alternatively, the shaft 231 of the ablation instrument 230 may comprise a wire system or other means to deflect the distal end of the ablation instrument 230 such that a distal end of the ablation instrument 230 does not damage the imaging transducer 107. In some embodiments, the ablation instrument 230 may rotate relative to the imaging component 100. In some embodiments, the ablation instrument 230 may fully rotate relative to the imaging component 100 in either direction under the control of a user within the shaft 103 of the imaging component 100 while the shaft 103 remains stationary, such that the tines 233 may be optimally aligned.

The needle assembly may be constructed and controlled by a user, for example, as previously described in commonly owned U.S. Pat. Nos. 8,206,300, 8,262,574, and 8,992,427, the full disclosures of which are incorporated herein by reference. The needle assembly may be integrated into an instrument handle such that the position and deployment of the introducer 235 and tines 233 may be controlled by the user. The handle may be constructed, for example, as previously described in commonly owned U.S. Pat. No. 8,992,427, the full disclosure of which is incorporated herein by reference. The needle assembly may be compatible with systems and methods for improved safety and treatment boundaries during the treatment of uterine fibroids as, for example, described in the incorporated references.

FIG. 2D shows a view of an imaging component 100 with a drug delivery instrument 240 disposed within the shaft 103 of the imaging component 100, in accordance with some embodiments. A drug delivery instrument 240 may serve as a platform to inject therapeutic agents into the tissue of a patient. Exemplary therapeutic agents may comprise analgesics, anesthetics, hemostatics, antibiotics, steroids, anticoagulants, anti-inflammatories, etc. Additionally or alternatively, the drug delivery instrument 240 may be configured to deliver to target tissue, one or more drug eluting, drug releasing, or otherwise therapeutic and/or diagnostic seeds, pellets, or other implants. The drug delivery instrument 240 may comprise needle 243 disposed inside a distal end of a shaft 241 of a drug delivery instrument 240. The shaft 241 of a drug delivery instrument 240 may comprise a distal end and a proximal end. The shaft 241 of the drug delivery instrument 240 may be longer than the shaft of the imaging transducer 107 such that the needle 243 may inject agents deep inside the uterus. In some embodiments, the shaft 241 of the drug delivery instrument 240 may be two inches longer than the shaft of the imaging transducer 107. Additionally or alternatively, for example, the shaft 241 of the drug delivery instrument 240 may be six inches longer, may be four inches longer, may be two inches longer, may be the same length, or may be within a range of any two of the preceding values.

The shaft 241 of drug delivery instrument 240 may be made of a pliable and/or flexible material such that it may be deflected by the imaging transducer 107 and/or an angled portion within the cavity 105 of the shaft 103 of the imaging component 100. Additionally or alternatively, the shaft 241 of the drug delivery instrument 240 may comprise a wire system or other means to deflect the distal end of the drug delivery instrument 240 such that a distal end of a drug delivery instrument 240 does not damage the imaging transducer 107. In some embodiments, the drug delivery instrument 240 may rotate relative to an imaging component 100. In some embodiments, the drug delivery instrument 240 may fully rotate relative to an imaging component 100 in either direction under the control of a user within the shaft 103 of the imaging component 100 while the shaft 103 of the imaging component 100 remains stationary.

The shaft 241 of a drug delivery instrument 240 may be detachable from a handle component of the instrument or may be constructed without a handle component such that the drug delivery instrument 240 may be disposable. In the illustrated embodiment, drug delivery instrument 240 does not have a handle portion. In such embodiments, the handle portion 101 of the imaging component 100 (shown, e.g., in FIG. 1A) may be used to guide the drug delivery instrument 240 during a procedure. Shown in FIG. 2D, the imaging component 100 may have a scale, a guide, or other indicia 245 on the inside face of the handle portion 109 in order to gauge the insertion depth of a needle 243 of a drug delivery instrument 240.

FIG. 2E shows a cross-sectional view of the imaging component 100 with a needle 243 disposed within the shaft 103 of the imaging component 100, in accordance with some embodiments. The shaft 241 of a drug delivery instrument 240 comprising a needle 243 may be disposed within a tube 115 disposed within the cavity 105 of the imaging component 100. Additionally or alternatively, the shaft 241 of a drug delivery instrument 240 may be disposed within the cavity 105 of the imaging component 100 without the use of a tube. While the shaft 241 of the drug delivery instrument 240 in the illustrated example may define a circular geometry, in other embodiments the shaft 241 of the drug delivery instrument 240 may be elliptical any other geometric shape such that the shaft 241 of the drug delivery instrument 240 may be inserted or removed from the cavity 105 of the imaging component 100. In some embodiments, the shaft 241 of the drug delivery instrument 240 may be asymmetrical to provide an axis for alignment of the instrument within the cavity 105 of the imaging component 100. In some embodiments, the drug delivery instrument 240 may rotate relative to the imaging component 100. In other embodiments, the drug delivery instrument 240 may fully rotate relative to the imaging component 100 in either direction under the control of a user within the tube 155 of the shaft 103 of the imaging component 100 while the shaft 103 of the imaging component remains stationary.

FIGS. 2A to 2E illustrate exemplary instruments which may be disposed within the shaft 103 of an imaging component 100, which examples are not intended to be limiting. Other examples may comprise a fluid infusion and/or aspiration instrument. A fluid infusion and/or aspiration instrument may comprise an instrument with a shaft comprising a lumen therein, configured to conduct a fluid to a tissue of a patient. A fluid infusion and/or aspiration instrument may deliver fluid to cool a tissue. Additionally or alternatively, a fluid infusion and/or aspiration instrument may deliver a fluid to clean a tissue. Additionally or alternatively, a fluid infusion and/or aspiration instrument may deliver a fluid to inflate a bodily cavity. A fluid infusion and/or aspiration instrument may deliver a solution and/or suspension comprising a therapeutic agent, such as an antiseptic, an anesthetic, an analgesic, an antibiotic, a steroid, etc. A fluid infusion and/or aspiration element may be integrated into any of the instruments described herein. Alternatively, a fluid infusion and/or aspiration element may comprise an instrument to be inserted and retracted as a step during a multi-instrument procedure.

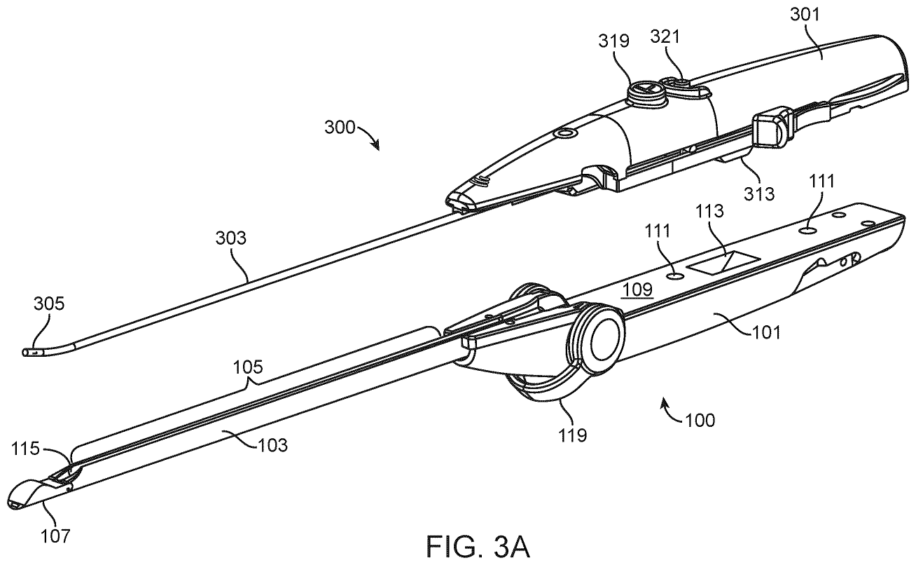

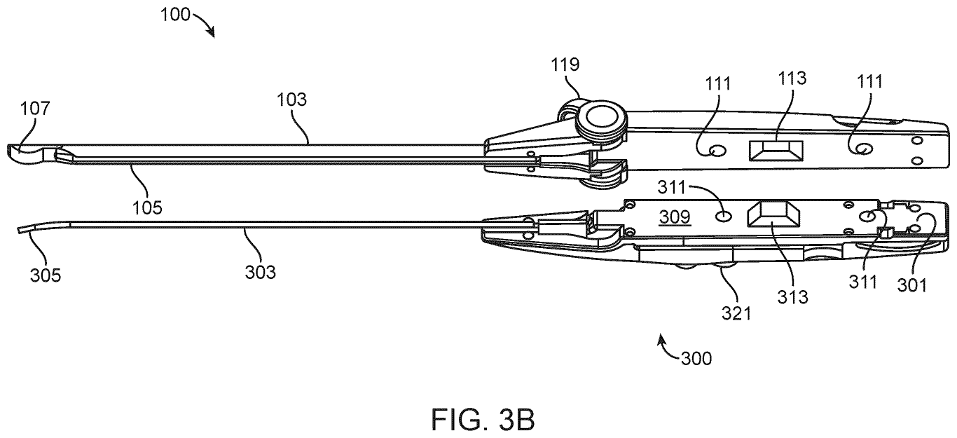

FIG. 3A shows an assembly view of an imaging system comprising an imaging component 100 and an optical scope instrument 300, in accordance with some embodiments. While an optical scope element may be shown in the illustrated embodiment, optical scope instrument 300 may be any other suitable instrument, for example, any of the instruments disclosed herein. Illustrated in FIG. 3A, the imaging system may slidably receive a disposable tube 115 within the cavity 105 of the imaging component 100. In some embodiments, the imaging system may comprise a disposable tube 115 slidably received within the cavity 105 of the imaging component 100. In such embodiments, an instrument may be removably received with a lumen of the disposable tube 115. Additionally or alternatively, the cavity 105 of the imaging component 100 may be configured to slidably receive one or more of a plurality of instruments, which instruments may comprise various therapeutic and/or diagnostic instruments.

In illustrative examples, the imaging component may removably receive an instrument such as a biopsy needle; a tissue collector instrument; an optical scope; implantation device; therapy electrodes; a tissue ablation element, such as for example a radiofrequency ablation element, an ultrasonic ablation element, a heat-based ablation element, a cryoablation element, etc.; and/or other instrument suitable to be disposed within the cavity of the imaging component. Additionally or alternatively, the instrument may be used to deliver drugs or other therapeutic agents to the tissue to be treated. Additionally or alternatively, with or without the use of a disposable tube, the imaging component may removably receive any of the instruments illustrated in FIG. 2A-2E.

In the illustrated embodiment, the distal end 305 of the optical scope instrument 300 may comprise a light emitting diode and/or a camera in order to provide images to a user. In such embodiments, the optical scope instrument 300 may serve as an endoscope. The distal end 305 of the optical scope instrument 300 may comprise optical components, such as an optic fiber, a relay lens, an objective lens, etc. The optical scope instrument 300 may comprise a shaft 303 of an optical scope instrument 300, which has a distal end and a proximal end. The shaft 303 of optical scope instrument 300 may be configured to detach from a handle component of the instrument or may be configured to be used without a handle component such that the optical scope instrument 300 may be disposable.

The shaft 303 of optical scope instrument 300 may be made of a pliable and/or flexible material such that it may be deflected by the imaging transducer 107 and/or an angled portion within the cavity 105 of the shaft 103 of the imaging component 100. Additionally or alternatively, the shaft 303 of the optical scope instrument 300 may comprise a (e.g., push, pull, and/or rotate/torque) wire system or other means to deflect the distal end 305 of the optical scope instrument 300. Deflection of a distal end 305 of an optical scope instrument 300 may serve to prevent damage to the imaging transducer 107 and/or allow multiple image angles may be collected. In some embodiments, the optical scope instrument 300 may rotate relative to the imaging component 100. In some embodiments, the optical scope instrument 300 may fully rotate relative to the imaging component 100 in either direction under the control of a user within the shaft 103 of the imaging component 100 while the shaft 103 of the imaging component remains stationary, such that multiple image angles may be collected.

The shaft 303 of the optical scope instrument 300 may be longer than the shaft of the imaging transducer 107 such that images may be collected from deep inside the uterus. In some embodiments, the shaft 303 of the optical scope instrument 300 may be two inches longer than the shaft of the imaging transducer 107. Additionally or alternatively, for example, the shaft 303 may be six inches longer, may be four inches longer, may be two inches longer, may be the same length, or may be within a range of any two of the preceding values.

In the illustrated embodiment, the optical scope instrument 300 comprises a handle portion 301. While a handle portion 301 may be shown connected to an optical scope in the illustrated example, similar handle portions may be connected to any suitable instrument, such as those disclosed herein. The handle portion 301 may be the second part of a two-part handle such that when an optical scope instrument 300 may be slidably inserted into the imaging component 100, the two handle portions may combine to form a single handle. The handle portion may additionally comprise a positioning element 313, in order to provide a more secure reference between parts of the two-part handle. Positioning element 313 may mate with positioning element 113. In such embodiments, the handle portion may comprise a release control 321, which may be actuated by a user, to retract the positioning element 313 into the handle and allow the two-part handle to be separated.

The handle portion 301 may additionally comprise one or a plurality of control elements 319. Control elements 319 may allow a medical professional to control the distal end of an instrument. In one example, the control element 319 controls a wire system which may reproducibly deflect or steer a distal end of an instrument. Additionally or alternatively, the control element 319 rotates a shaft of an instrument (e.g., a shaft 303 of an optical scope instrument 300) within the cavity 105 of the imaging component 100 or within the disposable tube 115. In another example, the control element scoops tissue in a tissue collector instrument. In another example, the control element 319 deploys a needle assembly comprising optional tines in an ablation instrument. Additionally or alternatively, the control element 319 begins the ablation procedure. In another example, the control element 319 applies pressure to inject a chemical though a drug delivery instrument. In another example, the control element 319 begins or ends image collection in an optical scope instrument.

FIG. 3B shows an assembly view of an imaging system illustrating an attachment mechanism of a system, in accordance with some embodiments. The inside 309 of the handle portion 301 may comprise alignment elements 311. Alignment elements 311 may be configured such that the optical scope instrument 300 may be reproducibly aligned with respect to the imaging component 100 after changing instruments. Additionally or alternatively, the alignment elements 311 may sufficiently secure the instrument and the imaging component 100 with respect to one another to use the two handle portions 101, 301 as a single handle. In some embodiments, the alignment elements 311 may comprise magnets. In other embodiments, the alignment elements 311 may comprise for example: latches, hooks, or any other mechanism suitable to removably combine a two-part handle. The inside 309 of the handle portion 301 may additionally comprise a positioning element 313, in order to provide a more secure reference between parts of the two-part handle. In such embodiments, the handle portion may comprise a release control 321, which may be actuated by a user, to retract the positioning element 313 into the handle and allow the two-part handle to be separated.

In some embodiments, a method of detecting or sensing the identification of removable instruments is provided when coupling the imaging component 100 and the removable instrument (e.g., the optical scope instrument 300). The imaging component 100 may include software to recognize the removable instrument and manage the interconnection between the imaging component 100 and removable instrument. The sensor or mechanism may be, by way of non-limiting examples, optical, RF, magnetic, biometric, electronic and mechanical IDs and readers. The method will ensure only qualified removable devices are received on the imaging device to ensure that only compatible devices may be used with the imaging component 100.

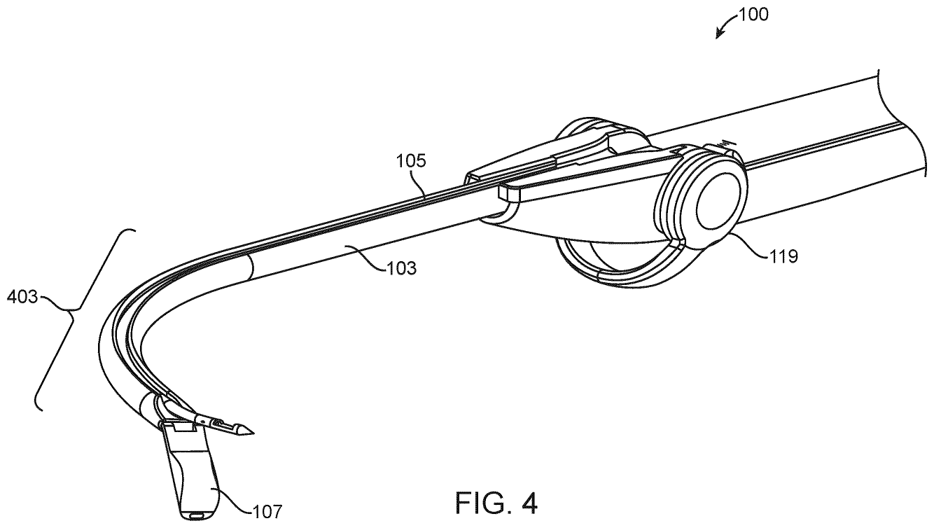

FIG. 4 illustrates a shaft 103 of an imaging component 100 wherein the shaft 103 of the imaging component 100 may be flexible, in accordance with some embodiments. In the illustrated embodiment, the shaft 103 of the imaging component 100 may comprise a flexible shaft portion 403. The body of the flexible shaft portion 403 may comprise internal structure in order to carry electronics or other associated components to control the imaging transducer 107. The imaging transducer 107 may comprise a channel or duct to direct fluid (e.g., water, saline, etc.) to a distal end of the shaft and onto a tissue surface. The flexible shaft portion 403 may comprise a fraction of the length of the shaft 103 of the imaging component 100. In some embodiments, the flexible shaft portion 403 comprises less than three-quarters the length of the shaft 103. Additionally or alternatively, the flexible shaft portion 403 may comprise less than a quarter the length of the shaft 103, and less than one eighth the length of the shaft 103, and the full length of the shaft 103.

The cross-sectional geometry of the flexible shaft portion 403 may continue the geometry of the shaft 103 such that no gaps or traumatic edges may be created between the flexible shaft portion 403 and the shaft 103. The flexible shaft portion 403 may be round in cross-section or take a shape with sufficiently softened, chamfered, rounded or beveled edges such that the edges may be atraumatic to a patient opening during insertion or removal of an imaging component 100 with or without an instrument. The flexible shaft portion 403 may additionally comprise a smooth exterior surface. The flexible shaft portion 403 may be made of a material such that the surface may be deformable to allow the flexible shaft portion 403 to bend or adapt to the shape of a bodily lumen.

The cavity of the flexible shaft portion 403 may be configured to slidably receive one or more of a plurality of instruments. The cavity of the flexible shaft portion 403 may be configured to continue the shape of the cavity 105 of the shaft 103 such that no gaps or traumatic edges may be created between the flexible shaft portion 403 and the shaft 103. In some embodiments, the cavity of the flexible shaft portion 403 may be partially open along a wall, such that a lumen of the cavity of the flexible shaft portion 403 may be in communication with the exterior of the shaft 103. The opening of the flexible shaft portion 403 may be sufficiently closed to provide structural support such that when the imaging component 100 may be inserted into a patient bodily lumen, the opening of the lumen may not be significantly disturbed by the insertion or removal of an instrument. In some embodiments, the edges of a cavity of the flexible shaft portion 403 may bend inward towards the interior of the cavity, such as in the embodiment illustrated in FIG. 1D. The inward bent edges of a cavity of the flexible shaft portion 403 may serve to support the opening of a bodily lumen such that the shaft 103 may be inserted or removed atraumatically from a bodily lumen with or without an instrument. The cavity of the flexible shaft portion 403 may be sufficiently open such that when instruments of different sizes may be received or inserted into the cavity, some distortion of the cavity opening may occur. The cavity may facilitate cleaning of the imaging component 100 by providing access to the interior of the cavity from its exterior.

While the cavity of the flexible shaft portion 403 in the illustrated example defines a circular cross sectional geometry, in other embodiments the cavity of the flexible shaft portion 403 may be elliptical or any other geometric shape with sufficiently softened, rounded, or beveled edges and corners such that insertion or removal of the shaft of the flexible shaft portion 403 does not damage the patient bodily lumen. In some embodiments, the cavity of the flexible shaft portion 403 may be asymmetrical to provide an axis for alignment of the instrument within. The cavity of the flexible shaft portion 403 may be open for less than three-quarters of its perimeter in cross-section, additionally or alternative, the cavity may be open for less than half its perimeter, less than a quarter its perimeter, and less than one eighth its perimeter. In other embodiments, the cavity of the flexible shaft portion 403 may be closed to the exterior of the shaft of the flexible portion, and an instrument may be slidably inserted fully interior to the shaft of the flexible portion.

In some embodiments, the flexible shaft portion 403 may be constructed from a pliable and/or flexible material such that it may be flexed within a patient bodily lumen. In some embodiments, the shaft may be controllably flexed along its longitudinal axis via a flex mechanism. Additionally or alternatively, the flexible shaft portion 403 may comprise a wire system or other flex mechanism in order to allow the flexible shaft portion 403 to controllably bend, flex, or deflect the distal end of the flexible portion. The flex mechanism may be controlled by a control element on a handle portion (e.g., handle portion 101, shown in FIG. 3A) of the imaging component 100.

In the illustrated example, the flexible shaft portion may be flexed axially to about a 90 degree angle with respect to the handle. Additionally or alternatively, the flexible shaft portion may be flexed axially to, for example, less than 180 degrees, less than 120 degrees, less than 90 degrees, less than 45 degrees, less than 10 degrees, less than 1 degrees. Additionally or alternatively, the flexible shaft portion may be flexed in an anterior-posterior axis relative to the handle of the imaging component 100. In some embodiments, the flexible shaft portion may be flexed in an anterior-posterior axis to, for example, less than 180 degrees, less than 120 degrees, less than 90 degrees, less than 45 degrees, less than 10 degrees, less than 1 degrees. Additionally or alternatively, the flexible shaft portion may be flexed in a medial-lateral axis relative the handle of the imaging component 100. In some embodiments, the flexible shaft portion may be flexed in a medial-lateral axis to, for example, less than 180 degrees, less than 120 degrees, less than 90 degrees, less than 45 degrees, less than 10 degrees, less than 1 degrees.

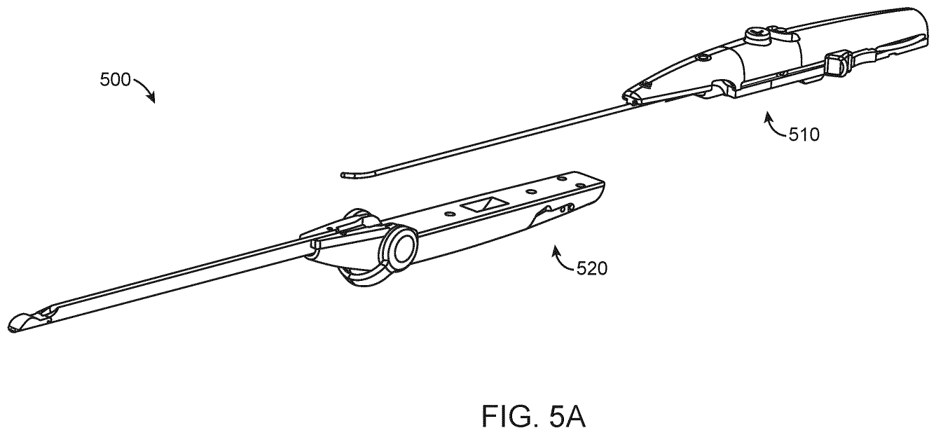

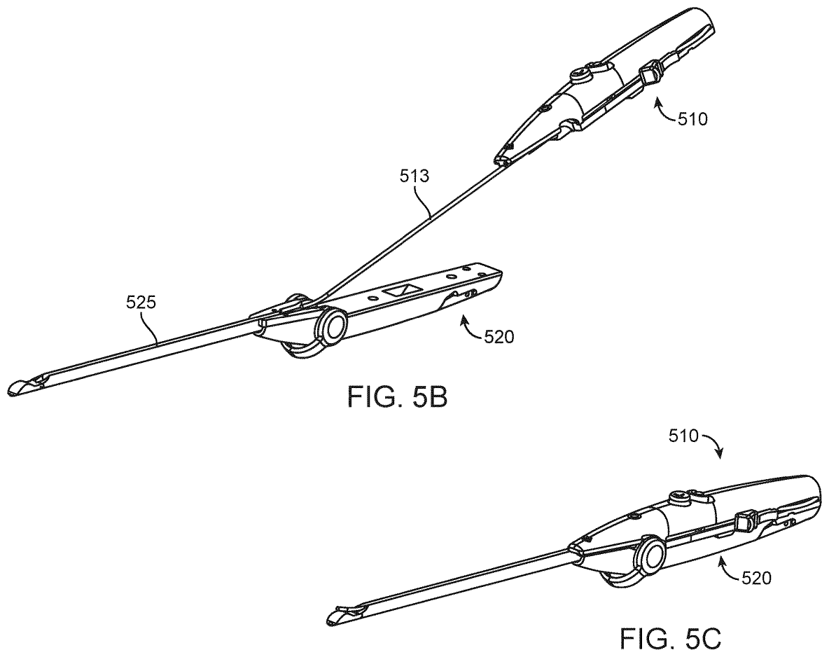

FIG. 5A illustrates a system for diagnosing and/or providing therapy, which may be removably coupled to multiple therapeutic and/or diagnostic instruments, in accordance with some embodiments. A system for performing therapy and/or diagnosis may comprise a therapeutic or diagnostic instrument 510 and an imaging component 520. An instrument 510 of the system for performing therapy and/or diagnosis may comprise a therapeutic or diagnostic instrument, such as, for example, any of the therapeutic or diagnostic instruments described herein. In some embodiments, the imaging component 520 may be used in conjunction with an instrument such as a biopsy needle; a tissue collector, an optical scope; implantation device; therapy electrodes; a tissue ablation element, such as for example a radiofrequency ablation element, an ultrasonic ablation element, a heat-based ablation element, a cryoablation element, etc.; and/or any other instrument suitable to be disposed within the cavity of the imaging component. Additionally or alternatively, the instrument may be used to deliver drugs or other therapeutic agents to the tissue to be treated. FIGS. 2A-2E shows exemplary instruments which may be slidably received by the imaging component. In some embodiments, the system may comprise a first and a second therapeutic or diagnostic instrument. An imaging component 520 may comprise an imaging component, such as, for example, examples, embodiments, and variations on the imaging component described herein.

FIG. 5B illustrates a system for diagnosing and/or providing therapy with a therapeutic and/or diagnostic instrument 510 being removably coupled to an imaging component 520, in accordance with some embodiments. As shown, the instrument 510 may be axially aligned with respect to the imaging component 520. Additionally, the distal end of the shaft 513 of the instrument 510 may be fed into the proximal end of the cavity 525 of the imaging component 520. Subsequently, the instrument 510 may be advanced toward the imaging component 520, such that the shaft 513 of the instrument 510 is slidably received by the cavity 525 of the imaging component 520. The instrument 510 may be slidably removed from the imaging component 520 by a similar procedure.

FIG. 5C illustrates a system for diagnosing and/or providing therapy with a therapeutic and/or diagnostic instrument 510 removably coupled to an imaging component 520, in accordance with some embodiments. The system for diagnosing therapy may comprise retention elements such as hooks, latches, or the mechanical features described herein in order to secure the instrument 510 to the imaging component 520. The system for diagnosing and/or providing therapy may be configured to couple to a plurality of instruments. For example, a first instrument may be coupled to an imaging component 520, and, subsequently, a second instrument may be coupled. The imaging component 520 may be configured to be coupled to both the first and second therapeutic and/or diagnostic instrument either simultaneously or individually. For example, if the first instrument is a disposable tube, the second instrument may be slidably inserted within the first instrument. In some embodiments, the imaging component 520 may be configured to be deliverable to the target site within the patient previously coupled with the first and/or second therapeutic or diagnostic instruments exterior to the target site. Additionally or alternatively, the imaging component 520 may be configured to be removably coupled to both the first and second therapeutic or diagnostic instruments, either simultaneously or individually, after the imaging component 520 is delivered to the target site within the patient (e.g., the instrument may be coupled in situ).

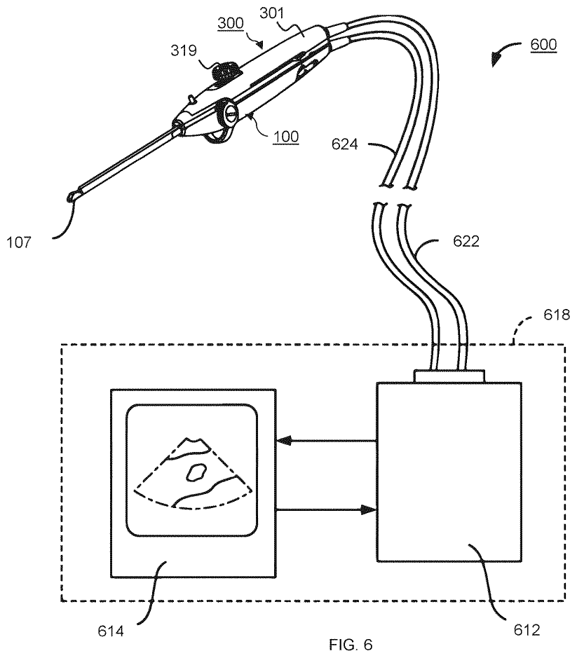

FIG. 6 shows an imaging system 600 comprising a digital processing device 612 and a display 614, the display 614 being visible to a user, in accordance with some embodiments. As illustrated in FIG. 6, an imaging system 600 may additionally comprise an imaging component 100 and an instrument 300. The digital processing device 612 may comprise one or more processors configured with instructions to set and record both treatment parameters and imaging parameters. The display 614 may be included in a common enclosure 618; however, in other embodiments, the display 614 may be remote to a digital processing device and/or the imaging component 100. The imaging component 100 may be connected to the digital processing device 612 by an imaging cord 624 to provide the image(s) captured by to the digital processing device 612 to be displayed by the display 614; however, additionally or alternatively, the imaging component 100 may communicate with the digital processing device 612 wirelessly. The instrument 300 may be connected to the digital processing device 612 by an instrument cord 622; however, additionally or alternatively, the instrument 300 may communicate with the digital processing device 612 wirelessly. In embodiments where the imaging component 100 and the instrument 300 are connected by cords 624, 622, the digital processing device 612 may supply power to both components.

The instrument 300 may comprise a handle portion 301 having one or more slidably mounted control elements 319 on its upper surface. In some embodiments, the control elements 319 may control the positioning of internal stops within the handle which may be monitored by the digital processing device 612 in order to calculate the size and position of the boundaries of the targeting region and/or the safety region which are shown on the display 614. In embodiments where instrument 300 is an ablation element (e.g., ablation instrument 230, shown in FIG. 2C), the stops may also serve to physically limit deployment of the introducer and optionally tines.

Some embodiments of the methods and systems of the present disclosure may be integrated with systems and methods for establishing and adjusting displayed safety and treatment zone boundaries. Such embodiments may include systems and methods of the incorporated references including: U.S. Pat. Pub. No. 2014/0073910 (now U.S. Pat. No. 9,861,336); U.S. Pat. No. 8,992,427; U.S. Pat Pub. No. 2018/0132927 (now U.S. Pat. No. 11,219,483); and P.C.T. Pub. No. WO2018/089523, the contents of which are incorporated herein by reference. Some embodiments of the methods and systems of the present disclosure may be integrated with systems and methods for mapping and planning systems. Such embodiments may include systems and methods of the incorporated references including P.C.T. Pub. No. WO2018/089523.

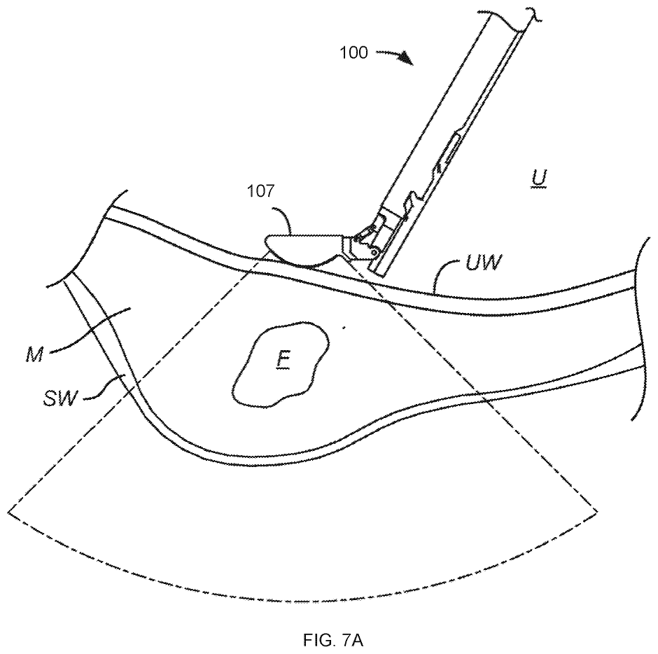

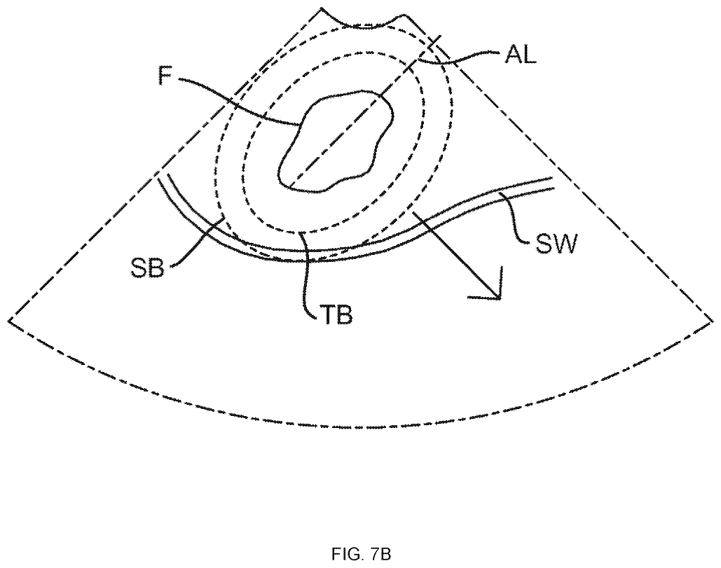

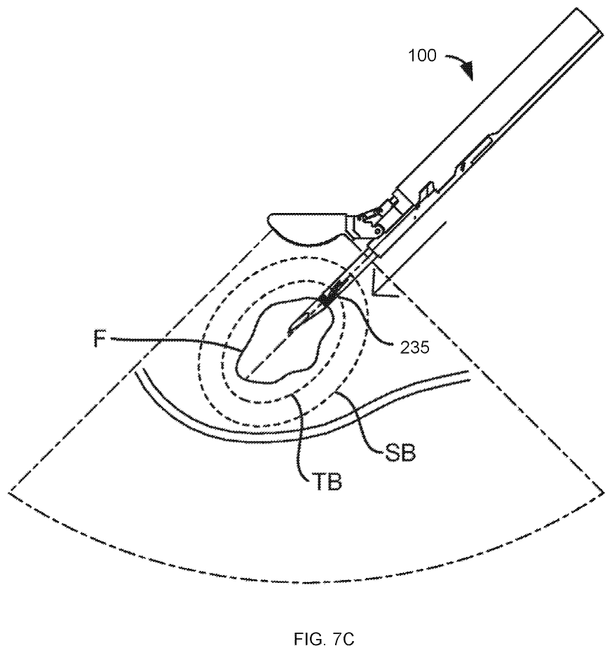

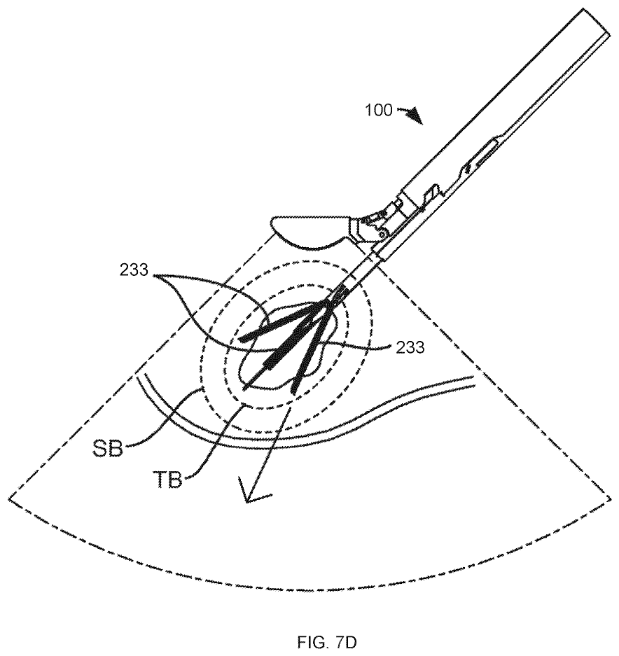

FIG. 7A illustrates an imaging component 100 which may be used to treat a fibroid F located in the myometrium M in a uterus U beneath a uterine wall UW (the endometrium) and surrounded by the serosal wall SW. The imaging component 100 can be introduced transvaginally and transcervically (or alternately laparoscopically) to the uterus U, and the imaging transducer 107 deployed to image the fibroid F within a field of view indicated by the broken lines.

FIG. 7B shows an image that would be visible on a display (e.g. display 614, shown in FIG. 6), showing safety and treatment boundaries, in accordance with some embodiments. In some embodiments, once the fibroid F is located on the display 614, the controls on the handle may be used to locate and size both a treatment boundary TB and a safety boundary SB. In some embodiments, initially, the virtual boundary lines TB and SB may neither be positioned over the fibroid F nor properly sized to treat the fibroid F. Prior to beginning therapy, the user (e.g., a physician) may want to both position and size the boundaries TB and SB for proper treatment. As the imaging transducer 107 may be already positioned against the uterine wall UW the only way to advance the treatment and safety boundaries TB, SB may be to move the boundaries forward by actuating the control element 319. In some embodiments, this may cause the treatment and safety boundaries TB and SB to move forwardly along the axis line AL and thereby translate the area to be treated. This may cause the virtual boundaries on the real-time image display 614 to move over the image of the fibroid F. Additionally or alternatively, the size of the treatment boundary TB may be enlarged or shrunk in order to mitigate the risk of affecting healthy and/or more sensitive tissue around the area of treatment.

In embodiments where the instrument is a tissue ablation element, while holding imaging component 100 steady, the physician may then advance a needle slide, causing the introducer 235 to extend into the fibroid F, as shown in FIG. 7C. The illustration in FIG. 7C includes a representation of the imaging component 100, which corresponds to the physical probe which is present in the patient. The remainder of FIG. 7C corresponds to the image present on the target display 614.

After introducer 235 has been fully deployed as limited by an optional physical or virtual needle stop housing in the instrument handle 301, the tines 233 may be deployed by advancing a tine slide. A target level of tine deployment is reached as indicated by engagement of the tine slide with an optional tine stop or visually on the display 614. Optionally, the imaging component 100 may be rotated about a central axis (typically aligned with the axis of the introducer 235) to confirm the treatment and safety boundaries TB, SB in all planes of view about the fibroid F. Display 614 will show the position of the treatment and safety boundaries TB, SB in real time relative to the target fibroid F and serosa. The tines 233 are then configured as shown in FIG. 7D, and power can be supplied to the tines 233 (and optionally to the introducer 235) in order to achieve treatment within the boundary depicted by the virtual treatment boundary TB. Again, FIG. 7D mixes both the virtual image which would be present on the display 614 as well as the physical presence of the imaging component 100.

### Uterine Fibroid Ablation Example

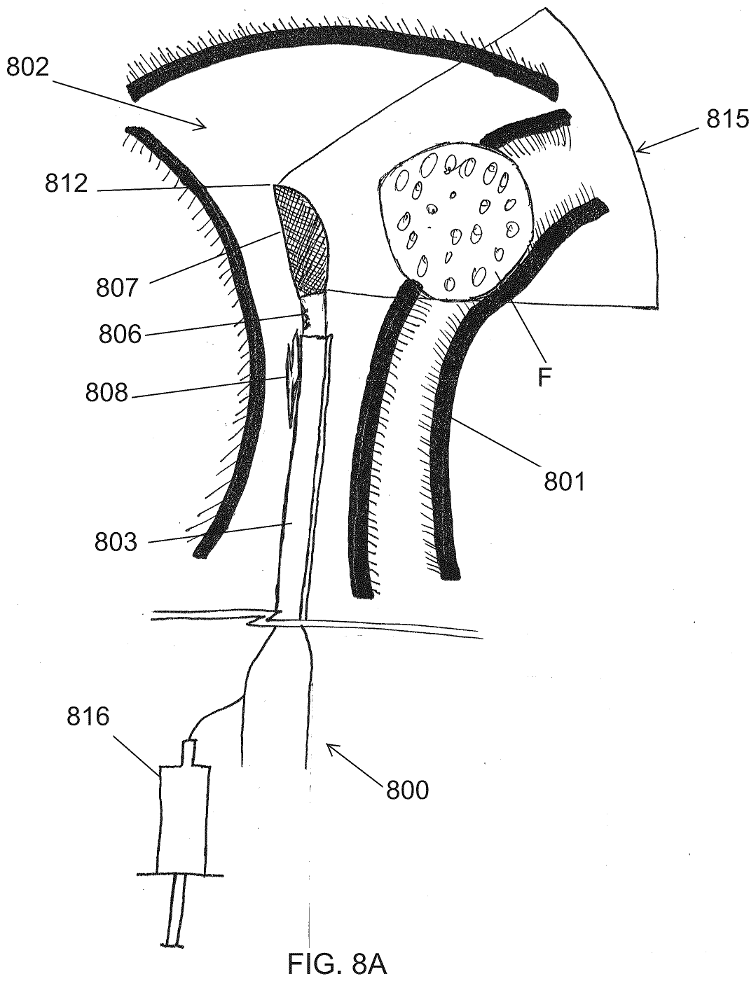

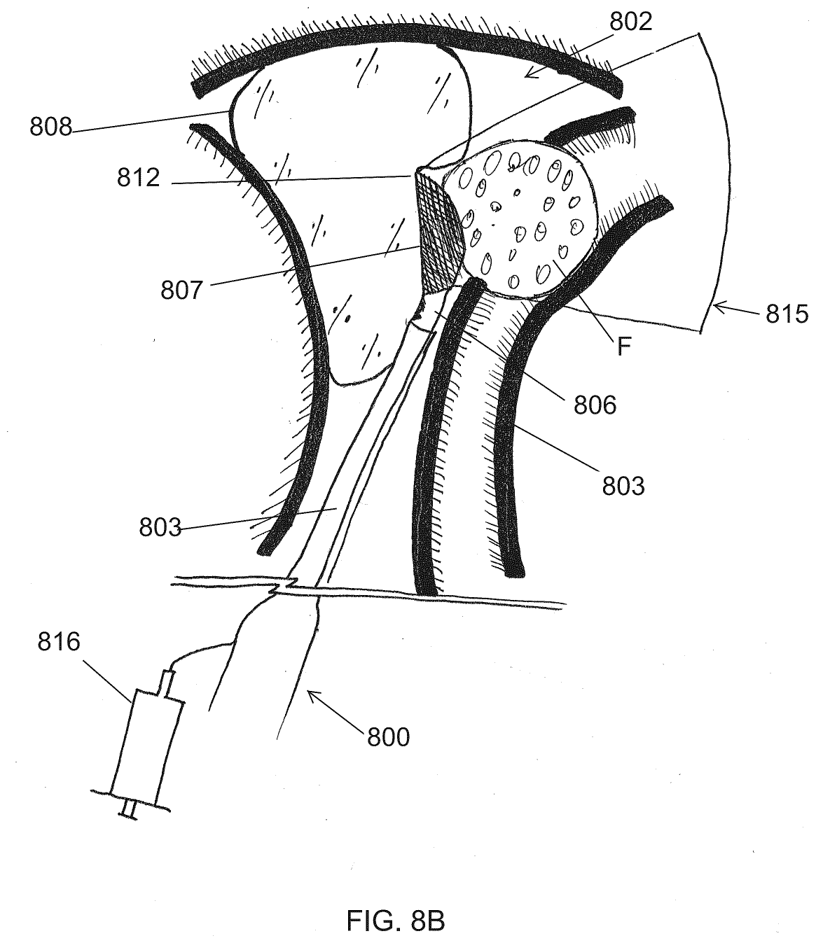

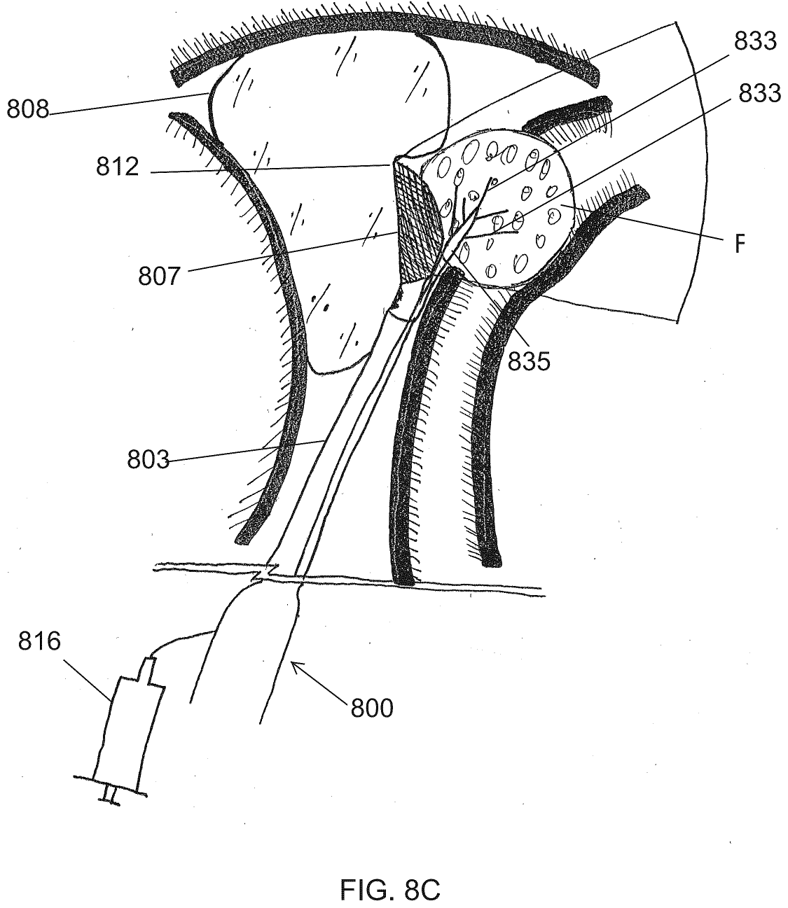

FIGS. 8A-8C depict an example of a system, device and method for uterine fibroid ablation. The device described herein may include an imaging component and a tissue ablation element as described above, for example a radiofrequency ablation element comprising an introducer and a plurality of needle electrodes or tines. The imaging component and the tissue ablation element may be coupled together for transvaginal and transcervical delivery into the uterus of a patient.

As illustrated, a tissue ablation device 800 is positioned within a uterine cavity 802. In this example the tissue ablation device 800 comprises an imaging component integrated into or coupled with a tissue ablation element, but in other examples the device 800 may comprise only an imaging component, a therapeutic component or other devices as described above. The device 800 can better articulate to access a sidewall of the uterus with the benefit of an expandable element 808. Each of FIGS. 8A-8C depict a fibroid F attached or otherwise at least partially embedded within a uterine wall 801.

In FIG. 8A, a tissue ablation device 800 is shown with a pivotable distal imaging transducer 807 and an expandable element 808 to articulate the ultrasound tip 812. The expandable element 808 as shown is in a reduced or collapsed configuration, according to embodiments disclosed herein. In FIG. 8A, a tissue ablation device 800 is shown within the uterine cavity 802. The tissue ablation device 800 comprises a device shaft 803. Provided at the distal end of the device shaft 803 is an imaging transducer 807, as described herein in embodiments above. Provided between the device shaft 803 and the imaging transducer 807 is a hinge 806. Provided at the distal tip of the imaging transducer 807 is an ultrasound tip 812. The tissue ablation device 800 further comprises an expandable element 808, which can be configured to articulate the device and/or ultrasound tip 812.

The device shaft 803 can comprise a straight shaft, which may be rigid or may have some flexibility. The device shaft 803 can comprise two attached subportions, such as an imaging shaft attached to a shaft of an instrument. One illustrative example of two attached subportions of a shaft is shown in FIG. 2C, in which a shaft 231 of an ablation instrument 230 is reversibly attached to an imaging shaft 103.

The imaging transducer 807 has a field of view 815 and is configured to image tissue (e.g., fibroid F) in a forward direction. The forward direction is defined as the direction in which the imaging transducer 807 is configured to image tissue.

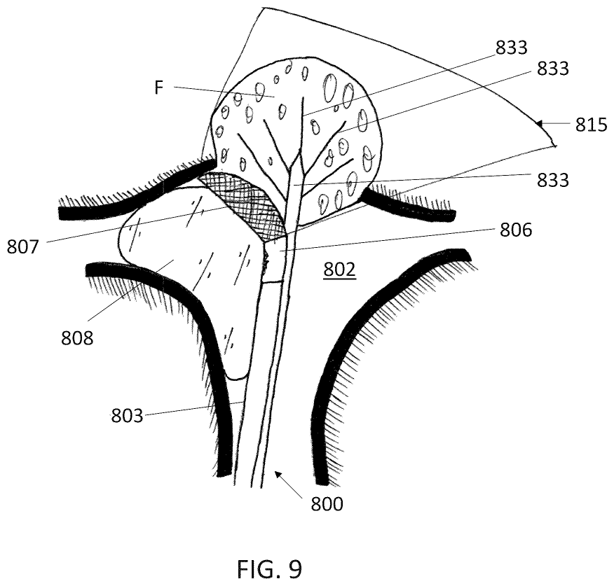

The hinge 806 permits the imaging transducer 807 to pivot relative to the device shaft 803, described herein. The imaging transducer 807 can reversibly pivot between a straight position and a pivoted position. The straight position is one in which the imaging transducer 807 forms a straight angle (i.e., roughly 180°) with the device shaft 803. A pivoted position, by contrast, is one in which the imaging transducer 807 is angled relative to the device shaft 803. An example of an imaging transducer 807 in a pivoted position can be seen in FIG. 8B and FIG. 9.

The ultrasound tip 812 of the tissue ablation device 800 can comprise a spring-loaded tip that is configured to apply a force to restore the imaging transducer 807 from a pivoted position toward the straight position. In some embodiments, the spring-loaded tip can be fully straight, or parallel and aligned with the device shaft 803, when the spring is not loaded. A spring-loaded tip can stabilize the imaging transducer 807 during placement and during an ablation procedure.

The expandable element 808 can be reversibly expanded from a reduced configuration (an example of which is shown in FIG. 8A) to an expanded configuration (examples of which are shown in FIGS. 8B and 8C). In some embodiments, the expandable element 808 can be directly coupled to the device shaft 803, either removably or integrally. The expandable element 808 can be coupled to the device shaft 803 proximal to the imaging transducer 807. Alternatively, the expandable element 808 can be coupled to the device shaft 803 such that a portion of the expandable element 808 is proximal to the imaging transducer 807 and another portion of the expandable element 808 is coupled to the imaging transducer 807. In some embodiments (such as in FIG. 8B), the expandable element 808 is configured to expand in a backwards direction, which is substantially opposite from the forward direction.

The expandable element 808 can comprise, for example, a balloon (as shown in FIGS. 8A-8C), a deployable push rod, an unwinding spring, or another kind of actuation design. A deployable push rod can be an expandable element 808 in some embodiments, for example, by being hingedly fixed to the device shaft: in a reduced or compressed configuration, the push rod can be folded down along the device, but in an expanded configuration, the push rod can be deployed outwardly to push against a wall of the uterus. In another embodiment, a deployable push rod can be attached to the device shaft and extend out of the side of the device shaft to push against a wall of the uterus. The expandable element 808 depicted in the examples of FIGS. 8A-8C and in FIG. 9 is a balloon 808. The shape, size, and/or materials of the balloon may be configured to control the size of the desired pressure from balloon inflation. In some embodiments of balloons used as an expandable element, the balloon may contain multiple chambers and each chamber can be controlled individually per procedural needs. In embodiments wherein the expandable element is a balloon, the balloon can be expanded by being filled with a filler fluid. The balloon may be inflated and deflated between targeting sequences. The tissue ablation device 800 can also comprise a reservoir 816 of filler fluid that is in fluid connection with the balloon. In some embodiments, the filler fluid enters the balloon, thereby expanding the balloon. In some embodiments, the filler fluid is a liquid, such as water or saline. In other embodiments, the filler fluid is gaseous, such as pressurized air, oxygen, nitrogen, or an inert gas such as helium or argon. In some embodiments, the reservoir 816 can be a syringe, a tank, or any other source of filler fluid. In FIGS. 8A-8C, the reservoir 816 is depicted as a syringe. In FIG. 8A, when the balloon 808 is in its reduced or collapsed configuration, the plunger of the syringe is fully drawn, indicating that the filler fluid of the system is in the syringe. In FIG. 8B, when the balloon 808 is in its expanded configuration, the plunger of the syringe is depressed, indicating that the filler fluid of the system has been moved from the syringe into the expanded balloon 808.

FIG. 8B shows the tissue ablation device 800 of FIG. 8A with its expandable element 808 (e.g., a balloon) in its expanded configuration, which can cause the tissue ablation device 800 to articulate (e.g., deflect or rotate) within the uterine cavity 802. The balloon in FIG. 8B is depicted as expanding in response to the filler fluid being forced from the reservoir 816 into the balloon. The pressure of the filler fluid can be adjusted through a sensor and control circuits. In some embodiments, the expandable element 808 can be configured to articulate the ultrasound tip 812. In some embodiments, the articulation action may be produced by expanding the expandable element 808 against the opposite wall of the uterine cavity 802 from the target fibroid F. Force against the opposite wall may stabilize the tissue ablation device 800 within the uterine cavity 802 by pushing against the opposite wall and filling the space of the uterine cavity 802. The balloon may be fixed to the back (non-transducer-facing side) of the device shaft 803 and proximal of the hinge 806 (as shown, e.g., in FIG. 8A). In some embodiments, as the balloon begins to take up space, the balloon pushes against the opposite wall and presses the device shaft 803 towards the fibroid F and also may articulate the spring loaded hinged transducer tip 812. In some embodiments, the expandable element is expanded before the tissue ablation device is placed at a target treatment area. Indeed, as shown in FIG. 8B, expanding the expandable element 808 can help place the tissue ablation device 800 at a target treatment area. In other embodiments (not shown), the expandable element can be expanded after the tissue ablation device is placed at the target treatment area.

In some embodiments, tissue ablation devices with expandable elements may be used to treat an endometrium surface by filling the balloon with high temperature filler fluid. In some embodiments, such a procedure can provide endometrial ablation, which can, for example, reduce or eliminate menstrual bleeding and accompanying symptoms. In some embodiments, the filler fluid can be a high temperature filler fluid. In some embodiments, the high temperature filler fluid can be heated to a temperature of about, at least about, or no more than about 75° C., 80° C., 85° C., 87° C., 90° C., 95° C., or more or less, and ranges including any two of the foregoing values for the duration of the endometrial ablation procedure.

In other embodiments (not shown), the expandable balloon may be positioned at the front of the imaging transducer and filled with a liquid. Configured as such, the expanded balloon may act as an acoustic standoff to couple the transducer with tissue automatically, improving ultrasound image quality during treatment procedures.

Returning to FIG. 8B, the expansion of the expandable element 808 (e.g., the balloon) against the opposite wall of the uterine cavity 802 can also act to press the imaging transducer 807 more closely against the fibroid F. In some embodiments, the force that acts to press the imaging transducer 807 more closely against the fibroid F can also cause the imaging transducer 807 to articulate at the hinge 806 relative to the device shaft 803 (or cause the device shaft 803 to articulate at the hinge 806 relative to the imaging transducer 807).

FIG. 8C shows the tissue ablation device 800 of FIG. 8B, in which an ablation instrument (e.g., the ablation instrument 230 in FIGS. 2C and 7C-7D) is inserted into the fibroid F for a treatment of fibroid ablation, as described herein. In some embodiments, the ablation instrument can be attached to the tissue ablation device 800 before the expandable element 808 is expanded. In other embodiments, the ablation instrument can be attached to the tissue ablation device 800 after the expandable element 808 is expanded. Hereinafter, it will be understood that the tissue ablation device 800 comprises an ablation instrument, which in turn comprises treatment elements, such as an introducer 835 and, optionally, a plurality of needle electrodes 833. As described herein, in some embodiments, the treatment elements can be inserted into the fibroid F. The manner in which the treatment elements are inserted into the fibroid F of FIG. 8C can be similar to those shown and described in, e.g., FIGS. 7C-7D. In some embodiments, articulating the imaging transducer 807 to a pivoted position can allow for deployment of the treatment elements. In FIG. 8C, the expandable element 808 can help provide additional stability to the introducer 835, to the needle electrodes 833, and to the imaging transducer 807 used to visualize the ablation procedure.

Providing balloon(s) or other expandable element as described herein can provide one or more benefits, including but not limited to: (1) stabilizing the ultrasound tip inside the cavity, (2) automatically pointing the device shaft at the target tissue or moving tissue into the line of sight for the introducer instead of requiring a deliberate pointing motion, (3) applying stabilization inside the uterus and tension in the tissue to reduce movement during introducer and needle electrode deployment, (4) creating stabilization during ablation, and (5) in conjunction with a spring loaded tip with live angle tracking, removing the need for the alignment step in the software.

Embodiments of the present disclosure are applicable to the Sonata® System available from Gynesonics, Inc. of Redwood City, CA and like systems, devices, and methods described in the following co-assigned U.S. Patents and Patent Applications, which are incorporated herein by reference: U.S. Pat. Nos. 7,918,795; 9,357,977; 7,815,571; 7,874,986; 10,058,342; 8,088,072; 8,206,300; 9,861,336; 8,992,427; 11,219,483; U.S. Pat. Pub. 2020/0275975 (now U.S. Pat. No. 11,612,431); and U.S. Pat. Pub. 2019/0350648.

FIG. 9 is similar to FIG. 8C, except in FIG. 9, the target tissue (e.g., fibroid F) is at the fundus of the uterine cavity 802, not along a side wall of the uterus. In some embodiments, the expandable element 808 (e.g., a balloon) in its expanded configuration can provide stability to the tissue ablation device 800 as it images and ablates fibroids at the fundus of the uterus. It will be understood by a person having ordinary skill in the art that the presence of an expandable element in its expanded configuration can help various procedures (e.g., imaging, ablation, biopsy, injection, etc.). It will also be understood that after a procedure, the expandable element 808 can be emptied or otherwise collapsed, returning the expandable element to its reduced configuration, which facilitates removal of the device from the uterus.

The foregoing description and examples has been set forth merely to illustrate the disclosure and are not intended as being limiting. Each of the disclosed aspects and embodiments of the present disclosure may be considered individually or in combination with other aspects, embodiments, and variations of the disclosure. In addition, unless otherwise specified, none of the steps of the methods of the present disclosure are confined to any particular order of performance. Modifications of the disclosed embodiments incorporating the spirit and substance of the disclosure may occur to persons skilled in the art and such modifications are within the scope of the present disclosure.

Terms of orientation used herein, such as “top,” “bottom,” “horizontal,” “vertical,” “longitudinal,” “lateral,” and “end” are used in the context of the illustrated embodiment. However, the present disclosure should not be limited to the illustrated orientation. Indeed, other orientations are possible and are within the scope of this disclosure. Terms relating to circular shapes as used herein, such as diameter or radius, should be understood not to require perfect circular structures, but rather should be applied to any suitable structure with a cross-sectional region that can be measured from side-to-side. Terms relating to shapes generally, such as “circular” or “cylindrical” or “semi-circular” or “semi-cylindrical” or any related or similar terms, are not required to conform strictly to the mathematical definitions of circles or cylinders or other structures, but can encompass structures that are reasonably close approximations.

Conditional language used herein, such as, among others, “can,” “might,” “may,” “e.g.,” and the like, unless specifically stated otherwise, or otherwise understood within the context as used, is generally intended to convey that some embodiments include, while other embodiments do not include, certain features, elements, and/or states. Thus, such conditional language is not generally intended to imply that features, elements, blocks, and/or states are in any way required for one or more embodiments or that one or more embodiments necessarily include logic for deciding, with or without author input or prompting, whether these features, elements and/or states are included or are to be performed in any particular embodiment.

Conjunctive language, such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z.

The terms “approximately,” “about,” and “substantially” as used herein represent an amount close to the stated amount that still performs a desired function or achieves a desired result. For example, in some embodiments, as the context may dictate, the terms “approximately”, “about”, and “substantially” may refer to an amount that is within less than or equal to 10% of the stated amount. The term “generally” as used herein represents a value, amount, or characteristic that predominantly includes or tends toward a particular value, amount, or characteristic. As an example, in certain embodiments, as the context may dictate, the term “generally parallel” can refer to something that departs from exactly parallel by less than or equal to 20 degrees.

Where term “about” is utilized before a range of two numerical values, this is intended to include a range between about the first value and about the second value, as well as a range from the first value specified to the second value specified.

Unless otherwise explicitly stated, articles such as “a” or “an” should generally be interpreted to include one or more described items. Accordingly, phrases such as “a device configured to” are intended to include one or more recited devices. Such one or more recited devices can be collectively configured to carry out the stated recitations. For example, “a processor configured to carry out recitations A, B, and C” can include a first processor configured to carry out recitation A working in conjunction with a second processor configured to carry out recitations B and C.

The terms “comprising,” “including,” “having,” and the like are synonymous and are used inclusively, in an open-ended fashion, and do not exclude additional elements, features, acts, operations, and so forth. Likewise, the terms “some,” “certain,” and the like are synonymous and are used in an open-ended fashion. Also, the term “or” is used in its inclusive sense (and not in its exclusive sense) so that when used, for example, to connect a list of elements, the term “or” means one, some, or all of the elements in the list.

Overall, the language of the claims is to be interpreted broadly based on the language employed in the claims. The language of the claims is not to be limited to the non-exclusive embodiments and examples that are illustrated and described in this disclosure, or that are discussed during the prosecution of the application.

Although systems, devices, and methods for endovascular implants and accurate placement thereof have been disclosed in the context of certain embodiments and examples, this disclosure extends beyond the specifically disclosed embodiments to other alternative embodiments and/or uses of the embodiments and certain modifications and equivalents thereof. Various features and aspects of the disclosed embodiments can be combined with or substituted for one another in order to form varying modes of systems, devices and methods for endovascular implants and accurate placement thereof. The scope of this disclosure should not be limited by the particular disclosed embodiments described herein.

Certain features that are described in this disclosure in the context of separate implementations can be implemented in combination in a single implementation. Conversely, various features that are described in the context of a single implementation can be implemented in multiple implementations separately or in any suitable subcombination. Although features may be described herein as acting in certain combinations, one or more features from a claimed combination can, in some cases, be excised from the combination, and the combination may be claimed as any subcombination or variation of any subcombination.

While the methods and devices described herein may be susceptible to various modifications and alternative forms, specific examples thereof have been shown in the drawings and are herein described in detail. It should be understood, however, that the invention is not to be limited to the particular forms or methods disclosed, but, to the contrary, the invention is to cover all modifications, equivalents, and alternatives falling within the spirit and scope of the various embodiments described and the appended claims. Further, the disclosure herein of any particular feature, aspect, method, property, characteristic, quality, attribute, element, or the like in connection with an embodiment can be used in all other embodiments set forth herein. Any methods disclosed herein need not be performed in the order recited. Depending on the embodiment, one or more acts, events, or functions of any of the algorithms, methods, or processes described herein can be performed in a different sequence, can be added, merged, or left out altogether (e.g., not all described acts or events are necessary for the practice of the algorithm). In some embodiments, acts or events can be performed concurrently, e.g., through multi-threaded processing, interrupt processing, or multiple processors or processor cores or on other parallel architectures, rather than sequentially. Further, no element, feature, block, or step, or group of elements, features, blocks, or steps, are necessary or indispensable to each embodiment. Additionally, all possible combinations, subcombinations, and rearrangements of systems, methods, features, elements, modules, blocks, and so forth are within the scope of this disclosure. The use of sequential, or time-ordered language, such as “then,” “next,” “after,” “subsequently,” and the like, unless specifically stated otherwise, or otherwise understood within the context as used, is generally intended to facilitate the flow of the text and is not intended to limit the sequence of operations performed. Thus, some embodiments may be performed using the sequence of operations described herein, while other embodiments may be performed following a different sequence of operations.

Moreover, while operations may be depicted in the drawings or described in the specification in a particular order, such operations need not be performed in the particular order shown or in sequential order, and all operations need not be performed, to achieve the desirable results. Other operations that are not depicted or described can be incorporated in the example methods and processes. For example, one or more additional operations can be performed before, after, simultaneously, or between any of the described operations. Further, the operations may be rearranged or reordered in other implementations. Also, the separation of various system components in the implementations described herein should not be understood as requiring such separation in all implementations, and it should be understood that the described components and systems can generally be integrated together in a single product or packaged into multiple products. Additionally, other implementations are within the scope of this disclosure.

Some embodiments have been described in connection with the accompanying figures. Certain figures are drawn and/or shown to scale, but such scale should not be limiting, since dimensions and proportions other than what are shown are contemplated and are within the scope of the embodiments disclosed herein. Distances, angles, etc. are merely illustrative and do not necessarily bear an exact relationship to actual dimensions and layout of the devices illustrated. Components can be added, removed, and/or rearranged. Further, the disclosure herein of any particular feature, aspect, method, property, characteristic, quality, attribute, element, or the like in connection with various embodiments can be used in all other embodiments set forth herein. Additionally, any methods described herein may be practiced using any device suitable for performing the recited steps.

The methods disclosed herein may include certain actions taken by a practitioner; however, the methods can also include any third-party instruction of those actions, either expressly or by implication. For example, actions such as “positioning an electrode” include “instructing positioning of an electrode.”

In summary, various embodiments and examples of endovascular implants and devices and methods for accurate placement have been disclosed. Although the systems, devices and methods for endovascular implants and accurate placement thereof have been disclosed in the context of those embodiments and examples, this disclosure extends beyond the specifically disclosed embodiments to other alternative embodiments and/or other uses of the embodiments, as well as to certain modifications and equivalents thereof. This disclosure expressly contemplates that various features and aspects of the disclosed embodiments can be combined with, or substituted for, one another. Thus, the scope of this disclosure should not be limited by the particular disclosed embodiments described herein, but should be determined only by a fair reading of the claims that follow.

The ranges disclosed herein also encompass any and all overlap, sub-ranges, and combinations thereof. Language such as “up to,” “at least,” “greater than,” “less than,” “between,” and the like includes the number recited. Numbers preceded by a term such as “about” or “approximately” include the recited numbers and should be interpreted based on the circumstances (e.g., as accurate as reasonably possible under the circumstances, for example ±5%, ±10%, ±15%, etc.). For example, “about 1 V” includes “1 V.” Phrases preceded by a term such as “substantially” include the recited phrase and should be interpreted based on the circumstances (e.g., as much as reasonably possible under the circumstances). For example, “substantially perpendicular” includes “perpendicular.” Unless stated otherwise, all measurements are at standard conditions including temperature and pressure.

What is claimed is:

1. A tissue ablation device comprising:
a device shaft;
an imaging transducer provided at a distal end of the device shaft providing an ultrasound tip; and
an expandable element configured to articulate the ultrasound tip.

2. The tissue ablation device of claim 1, wherein the imaging transducer is pivotable between a straight position in which the imaging transducer forms a straight angle with the device shaft, and a pivoted position in which the imaging transducer is angled relative to the device shaft.

3. The tissue ablation device of claim 2, wherein the ultrasound tip of the tissue ablation device comprises a spring-loaded tip, the spring-loaded tip configured to apply a force to restore the imaging transducer from a pivoted position toward the straight position.

4. The tissue ablation device of claim 1, wherein the expandable element is directly coupled to the device shaft.

5. The tissue ablation device of claim 1, wherein the imaging transducer is configured to image tissue in a forward direction, and wherein the expandable element is configured to expand in a backwards direction, the backwards direction being substantially opposite from the forward direction.

6. The tissue ablation device of claim 1, wherein the expandable element is a balloon.

7. The tissue ablation device of claim 6, wherein the balloon comprises a plurality of chambers, wherein each chamber can be controlled individually.

8. The tissue ablation device of claim 6, wherein the balloon is configured to be expanded by being filled with a filler fluid.

9. The tissue ablation device of claim 8, wherein the filler fluid is gaseous.

10. The tissue ablation device of claim 8, wherein the filler fluid is a high temperature filler fluid.

11. The tissue ablation device of claim 8, wherein the balloon is configured to expand between the imaging transducer and tissue being imaged by the imaging transducer, such that the balloon acts as an acoustic standoff to couple the imaging transducer with the tissue.

12. The tissue ablation device of claim 1, wherein the expandable element is coupled to the device shaft proximal to the imaging transducer.

13. The tissue ablation device of claim 1, wherein the expandable element is an unwinding spring.

14. The tissue ablation device of claim 1, wherein the device shaft comprises a straight shaft.

15. The tissue ablation device of claim 1, wherein the tissue ablation device is configured for uterine fibroid ablation.
